(19) United States
(12) Patent Application Publication
(10) Pub. No.: US 2025/0115942 A1
(43) Pub. Date: Apr. 10, 2025

(54) SCREENING METHOD FOR ANTI-PSEUDOMONAL AGENTS AND COMPOSITIONS AND METHODS FOR TREATING PSEUDOMONAS INFECTIONS

(71) Applicants: Matthew Thomas CABEEN, Stillwater, OK (US); Adam Stuart BRONSON, Richardson, TX (US); Nina Siobhan BAGGETT, Richardson, TX (US); Board of Regents for Oklahoma Agricultural and Mechanical Colleges, Stillwater, OK (US)

(72) Inventors: Matthew Thomas Cabeen, Stillwater, OK (US); Adam Stuart BRONSON, Richardson, TX (US); Nina Siobhan BAGGETT, Richardson, TX (US)

(21) Appl. No.: 18/695,480

(22) PCT Filed: Oct. 13, 2022
(86) PCT No.: PCT/US2022/078059

§ 371 Date: Mar. 26, 2024

Related U.S. Application Data
(60) Provisional application No. 63/255,147, filed on Oct. 13, 2021.

(51) Int. Cl. C12Q 1/18; C12N 1/20; C12Q 1/48

(52) U.S. Cl. CPC C12Q 1/18; C12N 1/20; C12Q 1/485; G01N 2333/91245

(57) ABSTRACT

Methods of screening for anti-pseudomonal compositions are disclosed that utilize a genetically engineered P. aeruginosa strain that is recA deficient and comprises a transcriptional reporter joined to at least a portion of a promoter for at least one gene of a pyocin gene cluster. The methods identify agents that stimulate pyocin expression in a RecA-independent manner. Also disclosed are agents identified by said methods, along with methods of producing and using same.

### CROSS-REFERENCE TO RELATED APPLICATIONS/INCORPORATION BY REFERENCE STATEMENT

This application claims benefit under 35 USC § 119(e) of U.S. Provisional Application Ser. No. 63/255,147, filed Oct. 13, 2021. The entire contents of the above-referenced patent application(s) are hereby expressly incorporated herein by reference.

### STATEMENT REGARDING FEDERALLY SPONSORED RESEARCH OR DEVELOPMENT

This invention was made with Government support under National Institute of General Medical Sciences Grant No. P20GM134973-01 and R35GM138018 awarded by the National Institutes of Health. The Government has certain rights in the invention.

### BACKGROUND

Pseudomonas aeruginosa is a gram-negative bacterial species that is ubiquitous in the environment. It is also infamous as a multidrug-resistant (MDR) opportunistic human pathogen most frequently associated with infections in immunocompromised populations. Pseudomonas aeruginosa is the leading cause of nosocomial infections and ventilator-associated pneumonia in the United States and is known to cause chronic pulmonary infections in the lungs of patients with cystic fibrosis (CF); as such, P. aeruginosa infection is associated with extremely high mortality rates in a variety of conditions. In addition, P. aeruginosa has developed numerous mechanisms of resistance to antibiotics, and a serious roadblock to treatment has been the lack of drugs inhibiting P. aeruginosa by new molecular mechanisms. Mechanisms of resistance to all known antibiotic agents have been identified in P. aeruginosa, leading the World Health Organization to declare P. aeruginosa as a “priority 1 critical pathogen” needing new strategies and options for prevention and chemotherapy (Kannon et al. (2022) Microbiology Spectrum, e02437-22).

P. aeruginosa is also known for aggressively competing with other bacterial cells using a variety of sophisticated offensive weapons. For instance, P. aeruginosa secretes soluble antibiotics like pyocyanin, enacts contact-mediated toxin injection using type-VI secretion, and can produce phage tail-like bacteriocins (PTLBs). Bacteriocins are typically named according to the producing species; in P. aeruginosa, they are termed pyocins and are thought to primarily enable intraspecies competition. There is also limited evidence that they may have activity against other species. Pyocins are thought to allow cells to exploit ecological niches through competition and domination.

Three types of pyocins are encoded by the P. aeruginosa genome; two of these, the R- and F-type pyocins, are PTLBs. The R-type pyocins are best understood with respect to their structure and mechanism; they are rod-shaped and comprise a contractile sheath surrounding a core component with an iron atom-tipped spike. R-type pyocins kill closely related cells by binding to pyocin subtype-specific lipopolysaccharides on the surface of the target cell and contracting. The effectiveness of R-type pyocins at killing target cells has raised interest in using these complexes as precision antimicrobials. F-type pyocins differ from R-type pyocins in both structure and mechanism. This type of pyocin is a filamented flexible rod that enacts killing via a noncontractile mechanism. In P. aeruginosa strains PAO1 and PA14, the R- and F-type pyocins are encoded in a region between trpE and trpG, with the R-type genes encoded first. The R/F pyocin gene cluster includes genes encoding a holin and a lysin, which function to perforate the cell membrane and digest the peptidoglycan cell wall, respectively, thereby releasing the relatively large R/F pyocins via lysis of producer cells.

The canonical pathway for pyocin expression is initiated by DNA damage, which activates RecA and also leads to the SOS response. Active RecA stimulates autocleavage of a repressor called PrtR, relieving repression of prtN, which encodes an activator of pyocin gene cluster expression. Fluoroquinolone antibiotics such as ciprofloxacin are commonly used in anti-pseudomonal therapy. They stabilize DNA-protein (topoisomerase IV and DNA gyrase) intermediates, leading to double-strand breaks, likely via multiple mechanisms. Accordingly, fluoroquinolones induce the SOS response and stimulate pyocin production. Previous reports indicate that SOS-induced pyocin production increases the susceptibility of P. aeruginosa to fluoroquinolones because of cell lysis induced by the holin and lysin proteins. Hence, pyocin production imposes a cost on a population of bacterial cells and can sensitize strains to antibiotic treatment.

Costly behaviors, such as sacrificial cell lysis to release bacteriocins, can be managed via heterogeneity so that such behaviors are enacted by only some cells in a population. Such heterogeneity has been observed for colicin production by Escherichia coli and has also been implicated in P. aeruginosa biofilms, where explosive lysis of a small subset of cells, mediated by the lysin gene of the R/F pyocin cluster, also functions to contribute extracellular DNA to biofilm communities. Microscopic observation of fluorescently tagged pyocin-like tailocins in P. protegens also revealed heterogeneity in tailocin production. Similarly, P. aeruginosa virulence appears to benefit from the lysis of a subset of cells mediated by the Alp pathway. A holin-encoding operon, alpBCDE, is activated by AlpA when repression by AlpR (which is homologous to PrtR) is relieved under DNA-damage conditions.

Given the significant morbidity and mortality associated with P. aeruginosa, as well as the multi-drug resistance thereof, there is a need in the art for new and improved anti-pseudomonal compositions, as well as methods of screening for and identifying same.

### BRIEF DESCRIPTION OF THE DRAWINGS

FIG. 1. Pyocin genes, but not canonical SOS genes, are upregulated in Δ69700, causing ciprofloxacin sensitivity. Panel A. Representative images of biofilm colonies grown on M6301 agar from which total RNA was extracted for transcriptomic analysis. RNA was extracted at day 4. Panel B. Volcano plot showing differentially regulated genes in ΔamrZ Δ69700 (MTC1398) versus the ΔamrZ parent (MTC590). A false-discovery rate (FDR) threshold of 10-3 was considered significant. R/F pyocin-encoding genes are shown in red, and the PA14 numbers of two other non-pyocin upregulated genes are labeled. Panel C. The same volcano plot as in panel B, showing in teal genes that were strongly upregulated under SOS-inducing conditions in Cirz et al. (19). Panel D. Representative growth curves of wild-type PA14 (MTC1) and Δ69700 (MTC1513) strains in LB-Lennox in the absence and presence of 0.03 μg/ml ciprofloxacin. Error bars show standard deviation of at least three technical replicates. Panel E. Indicator assays showing R/F pyocin production by PA14 and Δ69700; clearing indicates the presence of pyocins in cell-free culture supernatants.

FIG. 2. Pyocin gene upregulation and ciprofloxacin sensitivity in Δ69700 is caused by deficiency of xerC. Panel A. Schematic of the IppL operon showing the encoded enzymes and their relative expression (fold change) in ΔamrZ Δ69700 (MTC1398) versus ΔamrZ (MTC590). Panel B. Representative growth curves of PA14 (MTC1), ΔxerC (MTC2266), and ΔxerC attB::CTX-1-P_{IppL}-xerC (“attB::xerC”) (MTC2262) in LB-Lennox medium with and without 0.03 μg/ml ciprofloxacin. Panel C. Representative growth curves of PA14, Δ69700 (MTC1513) and Δ69700 attB::CTX-1-P_{IppL}-xerC (“attB::xerC”) (MTC2264) in LB-Lennox medium with and without 0.03 μg/ml ciprofloxacin. Error bars in Panels B and C show standard deviation of at least three technical replicates. Panel D. Indicator assays showing R/F pyocin production by PA14 compared to Δ69700 and ΔxerC strains complemented or not with xerC (attB::CTX-1-P_{IppL}-xerC). The supernatants were diluted twofold with fresh sterile LB where indicated.

FIG. 3. Deletion of xerC increases pyocin gene expression. Panel A. Representative transcriptional profile of a P₀₇₉₉₀-lux reporter (reporting on R/F pyocin gene transcription) during growth in LB-Lennox by wild-type PA14 (MTC2280), Δ69700 (MTC2281), or ΔxerC (MTC2297) strains. Panel B. Representative transcriptional profile as in panel B of wild-type PA14 or ΔxerC strains treated with 0.03 μg/ml ciprofloxacin. Panel C. Growth curves from the experiment shown in Panel B. Error bars show standard deviation of at least three technical replicates; some error bars are smaller than the graph symbols.

FIG. 4. Expression and production of pyocins by ΔxerC does not require RecA but requires PrtN. Panel A. Schematic of RecA-PrtN/R-mediated pyocin production. When the SOS response is inactive, PrtR represses prtN transcription so that pyocin expression is off. The presence of activated RecA during the SOS response induces PrtR autoproteolytic cleavage, permitting PrtN production and hence activating pyocin gene expression. Panel B. Representative transcriptional profiles (P₀₇₉₉₀-lux reporter) of PA14 (MTC2280), ΔxerC (MTC2297), and ΔxerC ΔrecA (MTC2301) strains. Panel C. Pyocin indicator assays using cell-free stationary-phase culture supernatants from PA14, ΔrecA (MTC2274), ΔxerC (MTC2266), and ΔxerC ΔrecA (MTC2288). The supernatants were diluted twofold with fresh sterile LB where indicated. D. Representative transcriptional profiles (P₀₇₉₉₀-lux reporter) of PA14, ΔxerC, and ΔxerC ΔprtN (MTC2298). Panel E. Pyocin indicator assays as indicated using cell-free stationary-phase culture supernatants from PA14, ΔprtN (MTC2276), ΔxerC (MTC2266), and ΔxerC ΔprtN (MTC2289) strains. Panel F. Pyocin indicator assay using cell-free stationary-phase culture supernatants of PA14, ΔxerC and their counterparts producing uncleavable PrtR_{5162A} as the only source of PrtR in the cell (MTC2305 and MTC2304, respectively). Panel G. Representative transcriptional profiles (P₀₇₉₉₀-lux reporter) of PA14 and ΔxerC strains without or with (MTC2308 and MTC2307, respectively) uncleavable PrtR_{5162A} as the only source of PrtR in the cell. Error bars in Panels B, D, and G show standard deviation of three technical replicates.

FIG. 5. Pyocin expression is heterogeneous across individual cells. Panel A. Representative phase-contrast and fluorescence micrographs of PA14 (MTC2277), ΔxerC (MTC2252), ΔxerC ΔrecA (MTC2291), and ΔxerC ΔprtN (MTC2292) cells growing on agarose pads. Panel B. Line histograms of average GFP fluorescence in individual cells of the indicated strains. Fluorescence is plotted as a multiple of the average background value, which is indicated with a black dashed line. The grey dashed line represents 1.2× the average background value, which was set as the threshold for GFP positivity (see Materials and Methods of Example 1 and FIG. 10). The number and percentage of positive cells is indicated. Panel C. Representative micrographs of PA14 pyocin-reporter cells treated in liquid culture for the indicated times with 1 μg/ml ciprofloxacin. Panel D. Line histograms of GFP mean fluorescence in individual cells of the indicated strains. Annotations are as in Panel B.

FIG. 6. Effect of XerC recombinase inactivation. Panel A. Sequence alignment of the C-terminal ends of E. coli MG1655 (SEQ ID NO:1) and P. aeruginosa PA14 XerC (SEQ ID NO:2_ proteins showing conservation (cyan boxes) and the catalytic Tyr residue (yellow). Panel B. Representative phase-contrast and fluorescence micrographs of PA14 xerC_{Y272F} bearing a P₀₇₉₉₀-gfp reporter at attB (MTC2341). Panel C. Line histograms of GFP mean fluorescence in individual PA14 xerC_{Y272F} cells (MTC2341). Panel D. Representative transcriptional profiles (P₀₇₉₉₀-lux reporter) of PA14 xerC_{Y272F} cells (MTC2339). Error bars show standard deviation of three technical replicates; some error bars are smaller than the symbols.

FIG. 7. Fate of cells that turn on pyocin expression. Panel A. Time-lapse series of fluorescence (GFP) and phase micrographs of PA14 ΔxerC cells bearing a P₀₇₉₉₀-gfp reporter at attB (MTC2252) to report on R/F pyocin expression. Asterisk indicates chained cells. White arrows denote cells that lyse before the next time point. Black arrow indicates a cell that shows no GFP expression but that lyses with the other cells in the microcolony. Panel B. Time-lapse series as in Panel A showing both a cell that strongly expresses GFP and lyses (white arrows) and cells that initially weakly express GFP but then appear to turn off pyocin expression and continue growing. Inset at 120 minutes is rescaled to show the relative weakness of GFP expression in cells that turn pyocin expression off compared to the strong expression of the cell that will lyse.

FIG. 8. Fate and phenotypes of pyocin-expressing cells deleted for lysis genes. Panel A. Time-lapse series of fluorescence (GFP) and phase micrographs of PA14 ΔxerC Δholin Δlysin cells bearing a P₀₇₉₉₀-gfp reporter at attB (MTC2293) to report on R/F pyocin expression. Arrows denote apparent cell death accompanied by loss of GFP fluorescence. Panel B. Representative GFP-phase contrast overlay of MTC2293 cells in exponential phase. Panel C. Representative growth curves of wild-type PA14 (MTC2280), ΔxerC (MTC2297), Δholin Δlysin (MTC2284) and ΔxerC Δholin Δlysin (MTC2299) grown in LB-Lennox medium. Panel D. Representative transcriptional profiles (P₀₇₉₉₀-lux reporter) of the same experiment as in Panel C. Panel E. Time-lapse series of fluorescence (GFP) and phase micrographs of PA14 ΔxerC Δpyocins cells bearing a P₀₇₉₉₀-gfp reporter at attB (MTC2332) to report on R/F pyocin expression. Arrows denote apparent cell death accompanied by loss of GFP fluorescence. Panel F. Representative growth curves of wild-type PA14 (MTC1), ΔxerC (MTC2266), Δpyocins (MTC2326) and ΔxerC Δpyocins (MTC2324) grown in LB-Lennox medium. Panel G. Manually curated plot of time intervals between appearance of visible GFP fluorescence and cell lysis or death (loss of GFP fluorescence) in ΔxerC (MTC 2252), ΔxerC Δholin Δlysin (MTC2293), and ΔxerC Δpyocins (MTC2324) cells (n=25 for each condition). Horizontal lines indicate the mean value for each condition. Italic letters denote P-values (2-tailed Student's t-test): a, 2.9×10⁻¹⁰; b, 2.0×10⁻¹²; c, 0.03. Panel H. Graph of cell debris produced by PA14 (MTC1), ΔxerC (MTC2266), Δholin Δlysin (MTC2295), and ΔxerC Δholin Δlysin (MTC2294) cells, as assayed by ultracentrifugation and FM4-64 staining of cell-free supernatants. Values shown are averages from three separate experiments, with error bars denoting standard deviation. Italic letters denote P-values (2-tailed Student's t-test): a, 0.0018; b, 0.0056. Error bars in Panels C-F indicate standard deviation of at least 3 technical replicates; some bars are smaller than the graph symbols.

FIG. 9. Effects of xerC deletion and complementation on colony morphology. Colony morphology after 6 days of growth on M6301 agar of the indicated strains. Wrinkled morphology is typically associated with biofilm formation.

FIG. 10. Determination of GFP fluorescence cutoff for positive cells. Line histogram of average per-cell fluorescence of PA14 cells containing no GFP reporter (MTC1) and imaged in the GFP channel as with GFP reporter. The fluorescence of 100% of reporter-less cells fell within 5 standard deviations of the mean value, and therefore 1.2× was chosen as the background value, which is between 5 and 6 standard deviations above the mean, as the cutoff for denoting cells as GFP-positive.

FIG. 11. Ciprofloxacin-mediated stimulation of pyocin expression in ΔxerC strains is RecA independent. Panel A. Representative growth curves (OD₆₀₀) and luminescence traces (P₀₇₉₉₀-lux) of wild-type PA14 (MTC2280) and ΔrecA (MTC2302) strains treated (grey) or not (black) with 0.03 μg/ml ciprofloxacin (Cipro). Note that the Y-axis scales on the luminescence graphs vary. Light grey shading surrounding the traces indicates standard deviation of three technical replicates. Time is indicated in hours. Panel B. Representative growth curves and luminescence traces as in Panel A, but for ΔxerC (MTC2297) and ΔxerC ΔrecA (MTC2301) strains. Panels C, D. OD-normalized luminescence traces of the indicated strains from Panels A and B, respectively.

FIG. 12. Mitomycin C-mediated stimulation of pyocin expression in ΔxerC strains is RecA independent. Panel A. Representative growth curves (OD₆₀₀) and luminescence traces (P₀₇₉₉₀-lux) of wild-type PA14 (MTC2280) and ΔrecA (MTC2302) strains treated (grey) or not (black) with 0.1 μg/ml mitomycin C (MMC). Note that the Y-axis scales on the luminescence graphs vary. Light grey shading surrounding the traces indicates standard deviation of three technical replicates. Time is indicated in hours. Panel B. Representative growth curves and luminescence traces as in Panel A, but for ΔxerC (MTC2297) and ΔxerC βrecA (MTC2301) strains. Panels C, D. OD-normalized luminescence traces of the indicated strains from Panels A and B, respectively.

FIG. 13. Mitomycin C-mediated stimulation of pyocin expression in wild-type and ΔxerC strains requires PrtN. Panel A. Representative growth curves (OD₆₀₀) and luminescence traces (P₀₇₉₉₀-lux) of wild-type PA14 (MTC2280) and ΔprtN (MTC2303) strains treated (grey) or not (black) with 0.1 μg/ml mitomycin C (MMC). Note that the Y-axis scales on the luminescence graphs vary. Light grey shading surrounding the traces indicates standard deviation of three technical replicates. Time is indicated in hours. Panel B. Representative growth curves and luminescence traces as in Panel A, but for ΔxerC (MTC2297) and ΔxerC ΔprtN (MTC2298) strains. Panels C, D. OD-normalized luminescence traces of the indicated strains from Panels A and B, respectively.

FIG. 14. Single-cell analysis of pyocin expression in ciprofloxacin-induced strains. Representative phase-contrast and GFP fluorescence (P₀₇₉₉₀-gfp) micrographs are shown in each panel above distributions of mean GFP fluorescence in individual cells of the indicated strains. As in previous work, cells above a cutoff of 1.2× (grey dashed line) the background fluorescence (black dashed line) were considered GFP-positive. In each panel, untreated cells are compared to the same strain treated with 0.03 μg/ml ciprofloxacin for 135 min. All micrographs are sized and scaled identically. Panel A. PA14 (MTC2277). Panel B. ΔrecA (MTC2448). Panel C. ΔxerC (MTC2252). Panel D. ΔxerC ΔrecA (MTC2291). Percentages and average fluorescence (X background) of GFP-positive cells are indicated. A larger number of cells was analyzed in strains expected to have a lower proportion of GFP-positivity to improve detection of rare GFP-positive cells.

FIG. 15. Pyocin expression in cells with catalytically inactive XerC is RecA dependent. Panel A. Representative growth curves (OD₆₀₀) and luminescence traces (P₀₇₉₉₀-lux) of xerC_{Y272F} (MTC2339) strains treated (grey) or not (black) with 0.03 μg/ml ciprofloxacin (Cipro) or 0.1 μg/ml mitomycin C (MMC). Note that the Y-axis scales on the luminescence graphs vary. Light grey shading surrounding the traces indicates standard deviation of three technical replicates. Time is indicated in hours. Panel B. Representative growth curves and luminescence traces as in panel A, but for xerC_{Y272F} ΔrecA (MTC2444) strains. Panels C, D. OD-normalized luminescence traces of the indicated strains from Panels A and B, respectively, together with other strains shown for reference.

FIG. 16. Hexapeptide recombinase inhibitors do not stimulate pyocin expression. Panel A. Representative growth curves (OD₆₀₀) and luminescence traces (P₀₇₉₉₀-lux) of wild-type PA14 (MTC2280) strains treated with the indicated concentrations of WRWYCR (SEQ ID NO:3) recombinase inhibitor peptide or with WKHYNY (SEQ ID NO:4), a control peptide with no inhibitor activity (blue shades). Cells were also treated with 0.03 μg/ml ciprofloxacin (Cipro) as a control (grey). Light grey shading surrounding the traces indicates standard deviation of three technical replicates. Time is indicated in hours. Panel B. Representative growth curves and luminescence traces as in Panel A, but for ΔxerC (MTC2297). Panels C, D. OD-normalized luminescence traces of the indicated strains and treatments from Panels A and B, respectively. Panel E. Representative phase-contrast and GFP fluorescence (P₀₇₉₉₀-gfp) micrographs of wild-type PA14 (MTC2277) cells treated or not with 50 μM WRWYCR inhibitor peptide for 135 min. Panel F. Distributions of mean GFP fluorescence in individual cells treated as in Panel E. As in previous work, cells above a cutoff of 1.2× (grey dashed line) the background fluorescence (black dashed line) were considered GFP-positive.

FIG. 17. Recombinase inhibitors do not stimulate pyocin expression at sub-inhibitory concentrations or in combination with ciprofloxacin. Panel A. Representative growth curves (OD₆₀₀) and luminescence traces (P₀₇₉₉₀-lux) of wild-type PA14 (MTC2280) strains treated with the indicated concentrations of WRWYCR recombinase inhibitor peptide or with WKHYNY, a control peptide with no inhibitor activity (blue shades). Cells were also treated with 0.03 μg/ml ciprofloxacin (Cipro) as a control (grey). Light grey shading surrounding the traces indicates standard deviation of three technical replicates. Time is indicated in hours. Panel B. OD-normalized luminescence traces of the indicated treatments from Panel A. Panel C. Representative growth curves (OD₆₀₀) and luminescence traces (P₀₇₉₉₀-lux) of wild-type PA14 (MTC2280) strains treated with 0.03 ag/ml ciprofloxacin alone (grey) or with the indicated concentrations of WRWYCR inhibitor or WKHYNY control peptides (blue shades). Light grey shading surrounding the traces indicates standard deviation of three technical replicates. Time is indicated in hours. Panels D, E. OD-normalized luminescence traces of the indicated treatments from Panel C. In Panel D, ciprofloxacin-treated ΔxerC cells (MTC2297) are shown as a reference for the degree of pyocin induction in the absence of XerC.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (i.e., on a molar basis it is more abundant than any other individual species in the composition), and in particular instances, a substantially purified fraction is a composition wherein the object species comprises at least about 50 percent (on a molar basis) of all macromolecular species present. Generally, a substantially pure composition will comprise more than about 80 percent of all macromolecular species present in the composition, such as (but not limited to) more than about 85%, 90%, 95%, and 99%. In certain particular (but non-limiting) embodiments, the object species is purified to essential homogeneity (contaminant species cannot be detected in the composition by conventional detection methods) wherein the composition consists essentially of a single macromolecular species.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent to a subject/patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of infection by or growth of microbes and/or opportunistic infections. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the disease/infection in conjunction with the compositions of the present disclosure. This concurrent therapy can be sequential therapy, where the patient is treated first with one composition and then the other composition, or the two compositions are given simultaneously.

The terms “administration” and “administering,” as used herein, will be understood to include all routes of administration known in the art. In addition, the compositions of the present disclosure (and/or the methods of administration of same) may be designed to provide delayed, controlled, or sustained release using formulation techniques which are well known in the art.

The term “pharmaceutically acceptable carrier or excipient” includes any carriers or excipients known in the art that may be utilized in accordance with the present disclosure. For example (but not by way of limitation), a physiologically compatible carrier (e.g., saline) that is compatible with maintaining the structure/activity of the active ingredient(s) when administered, and compatible with the desired mode of administration, may be utilized as the pharmaceutically acceptable carrier in accordance with the present disclosure. In addition, the active ingredient(s) may be mixed with excipients which are pharmaceutically acceptable and compatible with the active ingredient(s). Suitable excipients include, for example but not by way of limitation, water, saline, dextrose, glycerol, ethanol, and the like, or any combination thereof.

Turning now to the non-limiting embodiments of the inventive concepts, screening methods for identifying anti-pseudomonal compounds are disclosed, along with compositions and kits, as well as methods of producing and using said compositions and kits for treating Pseudomonas aeruginosa infections. The methods, compositions, and kits rely upon the ability of certain agents to stimulate pyocin expression in a RecA-independent manner. The screening methods to identify said anti-pseudomonal agents utilize a genetically engineered P. aeruginosa strain that is recA deficient and comprises a transcriptional reporter joined to at least a portion of a promoter for at least one gene of a pyocin gene cluster. The anti-pseudomonal agents identified by the methods disclosed herein cause P. aeruginosa cells to release increased quantities of P. aeruginosa-killing pyocins as they die, and may also render P. aeruginosa cells more antibiotic sensitive, thereby amplifying the bactericidal effect.

Certain gene deletions in the bacterium P. aeruginosa sensitize bacterial cells to fluoroquinolone antibiotics and greatly increase the expression and production of pyocins, which lyse the producing cells and also can kill pyocin-sensitive P. aeruginosa target strains. The use of compounds that inhibit the same proteins whose deletions sensitize P. aeruginosa cells to fluoroquinolones, either alone or in a combinatorial use with fluoroquinolones, results in enhanced bactericidal activity and increased pyocin production.

Certain non-limiting embodiments of the present disclosure are directed to a method of screening for at least one anti-pseudomonal composition. The method comprises the steps of: contacting at least one candidate agent with a P. aeruginosa strain to form a mixture, wherein the P. aeruginosa strain has been genetically engineered to be recA deficient and to comprise a transcriptional reporter joined to at least a portion of a promoter for at least one gene of a pyocin gene cluster (such as, but not limited to, the R/F pyocin gene cluster); culturing the mixture under conditions that allow for expression of the transcriptional reporter; and determining that the at least one candidate agent is a putative anti-pseudomonal agent when the expression of the transcriptional reporter is increased when compared to expression thereof in the absence of the at least one candidate agent.

Any candidate agents known in the art or otherwise contemplated herein may be screened in accordance with the present disclosure. The candidate agents may be naturally-occurring or synthetic compounds. The candidate agents may be isolated, singular compounds or part of a library or compound collection. Particular (but non-limiting) examples of candidate agents include small molecules. Non-limiting examples of compound sets utilized in accordance with the present disclosure are provided in the Examples.

Any P. aeruginosa strain known in the art or otherwise disclosed herein may be utilized in accordance with the present disclosure, so long as the strain is recA deficient and comprises a transcriptional reporter joined to at least a portion of a promoter for at least one gene of a pyocin gene cluster (such as, but not limited to, the R/F pyocin gene cluster). In addition, in certain particular but non-limiting embodiments, the P. aeruginosa strain has been genetically engineered to inhibit pyocin production (such as, but not limited to deletion of at least one of PA14_07990 and PA14_08160) such that pyocin-mediated lysis is blocked, and/or has been genetically engineered to be alpBCDE deficient. Use of a P. aeruginosa strain genetically engineered in this manner prevents pyocin-mediated cell death and maximizes the sensitivity of the screen.

The P. aeruginosa strains utilized in accordance with the present disclosure may be genetically engineered to delete or inactivate one or more genes and to add a transcriptional reporter using genetic engineering methods that are well known in the art and widely commercially available. Particular non-limiting examples of genetically engineered P. aeruginosa strains that may be utilized in accordance with the present disclosure, as well as construction and production thereof, are disclosed in the Examples and in Tables 1-2, as well as in Baggett et al. (mBio (epub 2021 Nov. 23) 12(6):e0289321; doi: 10.1128/mBio.02893-21; PMID: 34809462; PMCID: PMC8609362) and Bronson et al. (Microbiol. Spectr. (epub 2022 Jun. 16) 10(4):e0116722; doi: 10.1128/spectrum.01167-22). The entire contents of these two references are hereby expressly incorporated herein by reference.

One particular non-limiting example of a genetically engineered P. aeruginosa strain that may be utilized in accordance with the present disclosure is P. aeruginosa PA14 ΔrecA attB::CTX-1-P₀₇₉₉₀-lux.

Any transcriptional reporters known in the art or otherwise contemplated herein that can be utilized in a screening assay format and that emits a quantitatively measurable signal can be utilized in accordance with the methods of the present disclosure. In certain particular (but non-limiting) embodiments, the transcriptional reporter is at least one of a luminescent compound and a fluorescent compound. Non-limiting examples of transcriptional reporters that can be utilized in accordance with the present disclosure include Green Fluorescent Protein (GFP), Yellow Fluorescent Protein (YFP), Red Fluorescent Protein (RFP), Cyan Fluorescent Protein (CFP), mCherry, mKate2, mPlum, a luciferase (such as, but not limited to, Firefly, Renilla, and Gaussia luciferases), and the like, as well as any combinations thereof.

In certain particular (but non-limiting) embodiments of the method, the mixture of candidate agent with genetically engineered P. aeruginosa strain is cultured in the presence of at least one DNA-damaging agent, such as (but not limited to) mitomycin C. The use of DNA-damaging agents in the culture may be useful when XerC is inactivated in the P. aeruginosa strain.

The method of the present disclosure may include one or more additional steps that further aid in determining whether a putative candidate agent possesses a desired activity. In a particular (but non-limiting) embodiment, the method further comprises the step of assaying the mixture to determine if at least one of tyrosine recombinase XerC or XerD expression or activity is inhibited when compared to expression or activity thereof in the absence of the at least one candidate agent.

In certain particular (but non-limiting) embodiments, the method is a high throughput screening (HTS) method.

Certain non-limiting embodiments of the present disclosure are directed to any of the genetically engineered P. aeruginosa strains constructed for use in the methods described herein above. The P. aeruginosa strains of the embodiments of the present disclosure are recA deficient and comprise a transcriptional reporter joined to at least a portion of a promoter for at least one gene of a pyocin gene cluster (such as, but not limited to, the R/F pyocin gene cluster). In addition, in certain particular but non-limiting embodiments, the P. aeruginosa strain has been genetically engineered to inhibit pyocin production (such as, but not limited to deletion of at least one of PA14_07990 and PA14_08160) such that pyocin-mediated lysis is blocked, and/or has been genetically engineered to be alpBCDE deficient. One non-limiting example of a genetically engineered P. aeruginosa strain in accordance with the present disclosure is P. aeruginosa PA14 ΔrecA attB::CTX-1-P₀₇₉₉₀-lux. Other non-limiting examples of genetically engineered P. aeruginosa strains in accordance with the present disclosure are disclosed in the Examples and in Table 2, as well as in Baggett et al. and Bronson et al. (incorporated supra).

Certain non-limiting embodiments of the present disclosure are directed to kits that comprise at least one of any of the genetically engineered P. aeruginosa strains disclosed or otherwise contemplated herein. In certain particular (but non-limiting) embodiments, the kits may further include one or more additional reagents or components for performing the screening methods described herein. The nature of these additional reagent(s) will depend upon the particular assay format, and identification thereof is well within the skill of one of ordinary skill in the art; therefore, no further description thereof is deemed necessary. Also, the compositions/reagents present in the kits may each be in separate containers/compartments, or various compositions/reagents can be combined in one or more containers/compartments, depending on the reactivity and stability of the compositions/reagents. For example (but not by way of limitation), the kit may further include positive and/or negative control reagents (such as, but not by way of limitation, any of the positive and/or negative controls described in the Examples). In addition, the kit may further include a set of written instructions explaining how to use the kit. A kit of this nature can be used in any of the methods described or otherwise contemplated herein.

Certain non-limiting embodiments of the present disclosure are directed to a composition that comprises at least one anti-pseudomonal agent identified by any of the screening methods disclosed or otherwise contemplated herein.

In certain particular (but non-limiting) embodiments, the composition may further include at least one fluoroquinolone antibiotic. Any fluoroquinolone antibiotics known or suspected to have anti-pseudomonal activity may be included in the composition in accordance with the present disclosure. Non-limiting examples of fluoroquinolones that may be utilized in accordance with the present disclosure include balofloxacin, besifloxacin, cinoxacin, ciprofloxacin, clinafloxacin, danofloxacin, delafloxacin, difloxacin, enoxacin, enrofloxacin, fleroxacin, flumequine, garenoxacin, gatifloxacin, gemifloxacin, garenoxacin, grepafloxacin, ibafloxacin, levofloxacin, lomefloxacin, marbofloxacin, moxifloxacin, nadifloxacin, naldixic acid, nemonoxacin, norfloxacin, ofloxacin, orbifloxacin, oxalinic acid, pazufloxacin, pefloxacin, piromidic acid, pipemidic acid, prulifloxacin, rosoxacin, rufloxacin, sarafloxacin, sitafloxacin, sparfloxacin, temafloxacin, tosufloxacin, trovafloxacin, DX-619; a pharmaceutically acceptable salt or stereoisomer of any of the above; or any combination thereof. Particular (but non-limiting) examples of fluoroquinolone antibiotics that may be utilized include ciprofloxacin, delafloxacin, gemifloxacin, levofloxacin, moxfloxacin, ofloxacin, or any combination thereof.

Certain non-limiting embodiments of the present disclosure are directed to a pharmaceutical composition that comprises at least one of any of the compositions containing at least one anti-pseudomonal agent identified by any of the screening methods disclosed or otherwise contemplated herein, in combination with a pharmaceutically-acceptable carrier or excipient. The at least one anti-pseudomonal agent may be utilized alone; alternatively, the composition may further include at least one of any of the fluoroquinolones disclosed or otherwise contemplated herein.

Certain non-limiting embodiments of the present disclosure are directed to a kit that comprises at least one of any of the compositions containing at least one anti-pseudomonal agent identified by any of the screening methods disclosed or otherwise contemplated herein, either alone or in combination with one or more additional reagents or components. For example (but not by way of limitation), the kit may further include at least one therapeutic agent, such as (but not limited to) any of the fluoroquinolones disclosed or otherwise contemplated herein. In another non-limiting example, the kit may further include at least one DNA-damaging agent, such as (but not limited to) mitomycin C. Such DNA-damaging agents, while not therapeutically useful, might nonetheless sensitize the screening methods disclosed or otherwise contemplated herein (especially if XerC is inactivated).

In certain particular (but non-limiting) embodiments, the kits may further include one or more additional reagents or components for performing the therapeutic methods described herein. The nature of these additional component(s) and/or reagent(s) will depend upon the particular administration format, and identification thereof is well within the skill of one of ordinary skill in the art; therefore, no further description thereof is deemed necessary. Also, the compositions/reagents present in the kits may each be in separate containers/compartments, or various compositions/reagents can be combined in one or more containers/compartments, depending on the reactivity and stability of the compositions/reagents. In addition, the kit may further include a set of written instructions explaining how to use the kit. A kit of this nature can be used in any of the methods described or otherwise contemplated herein.

Certain non-limiting embodiments of the present disclosure are directed to a method of treating a bacterial infection (such as, but not limited to, a P. aeruginosa infection) in a subject. The method includes administering to the subject at least one of any of the compositions disclosed or otherwise contemplated herein. The composition may include the at least one anti-pseudomonal agent utilized alone; alternatively, the composition may further include at least one of any of the fluoroquinolones disclosed or otherwise contemplated herein.

Alternatively, the method may include the separate administration of the at least one anti-pseudomonal agent-containing composition and the at least one fluoroquinolone. When administered separately, the two compositions may be administered substantially simultaneously or wholly or partially sequentially. When administered wholly or partially sequentially, the at least one anti-pseudomonal agent-containing composition may be administered before or after the at least one fluoroquinolone. In addition, the administration step(s) may be repeated one or more times.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

### Example 1

In this Example, a previously unknown pathway for strong pyocin expression that is independent of RecA and unconnected to the SOS response is uncovered. This pathway is active when levels of the recombinase XerC are lowered or when XerC recombinase activity is abrogated and is strongest when XerC is absent. Further, ΔxerC strains were analyzed at the single-cell level to visualize the distribution and dynamics of pyocin expression. Striking heterogeneity was observed, with a subset of cells progressively increasing pyocin expression before explosively lysing, implying a protective regulatory mechanism that commits only some cells to pyocin production and prevents widespread cell lysis. The entire contents of Baggett et al. (mBio (2021) 12(6):e0289321) are hereby expressly incorporated herein by reference in this Example.

### Results

### Deletion of PA14_69700 Results in Substantial Upregulation of Pyocin Genes

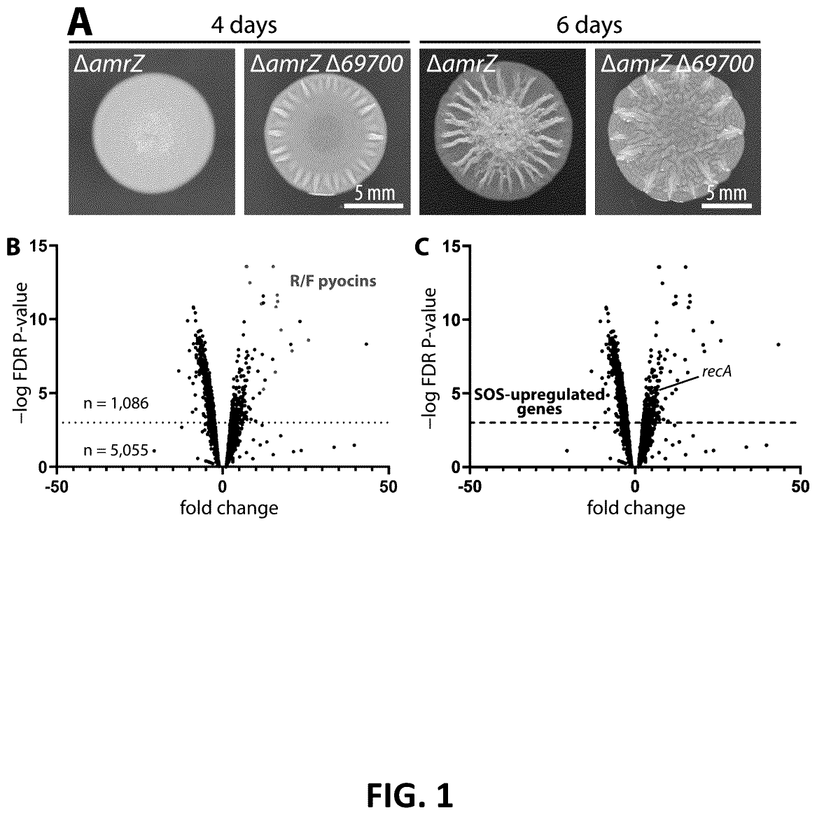

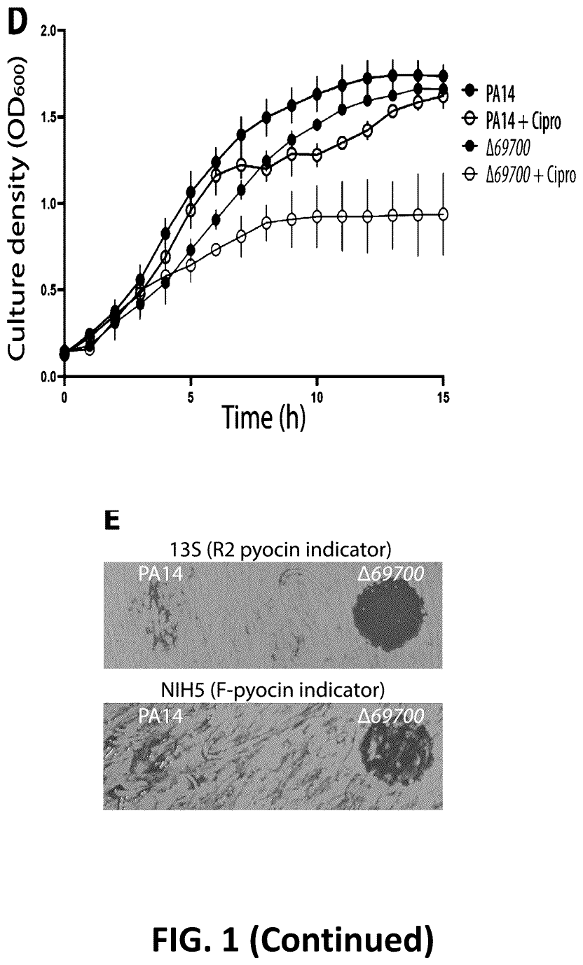

In previous work, the inventors showed that an in-frame deletion of an uncharacterized gene, PA14_69700 (also known as PA14_RS28410), resulted in enhanced biofilm formation by P. aeruginosa (Cabeen et al. (2016) Mol Microbiol, 99:557-70). To understand the mechanism by which 69700 deletion modulates biofilm formation, transcriptomic analysis was performed. RNA was extracted from 69700⁺ and Δ69700 biofilm colonies in the moderately hyper-wrinkled ΔamrZ genetic background from which the 69700 gene was first identified. It was anticipated that one or more genes responsible for the hyper-biofilm phenotype of 69700-deleted strains would be found (FIG. 1, Panel A). Deletion of 69700 resulted in a significant differential regulation of more than a sixth (n=1,062/6,142) of the entire P. aeruginosa PA14 genome (FIG. 1, Panel B). The differentially regulated genes are diverse in function: they include genes involved in metabolism, secretion systems (T2SS and T3SS), efflux, transcriptional regulation, and numerous genes whose functions remain unknown. However, there was a conspicuous group of adjacent, strongly upregulated genes (with log₂ fold changes of ˜2-5) between PA14_07970 and PA14_08300. These genes encode the R/F pyocins (FIG. 1, Panel B), which are phage tail-like, narrow-spectrum antimicrobial protein complexes produced by P. aeruginosa to kill closely related strains of the same species. Both the R and F types were highly upregulated. Given that deletion of 69700 increased the expression of R/F pyocins, it was sought to characterize its regulatory role further.

### Hallmark SOS Genes are not Activated by the 69700 Deletion

Canonical activation of the R/F pyocin gene cluster occurs following DNA damage and requires RecA activation, which also stimulates the SOS response. The SOS response regulates numerous genes, including genes involved in DNA repair and in metabolism. One trivial explanation for the observed pyocin gene upregulation is that the 69700 deletion activated the SOS response. However, among the set of known SOS-responsive genes in P. aeruginosa, none except recA exceeded the significance threshold (FIG. 1, Panel C). These data imply that the 69700 deletion does not induce pyocin production by stimulating the SOS response, indicating the existence of an alternate regulatory pathway for pyocin gene expression.

### The 69700 Deletion Mutant Shows Fluoroquinolone Sensitivity and Other Pyocin-Related Phenotypes

Previous reports suggested that pyocins are a determinant of sensitivity to fluoroquinolone antibiotics, prompting the examination of the fluoroquinolone sensitivity of Δ69700. Growth-curve experiments were performed in the presence and absence of sub-inhibitory concentrations of ciprofloxacin, which showed that both WT and Δ69700 cells grew to an OD₆₀₀ over 1.6. In the presence of ciprofloxacin (0.03 μg/ml), WT cells showed slower growth en route to a slightly lower final OD, while Δ69700 cells only reached an OD₆₀₀ of only 0.8 (FIG. 1, Panel D), consistent with increased ciprofloxacin susceptibility correlating with elevated pyocin production in Δ69700. To confirm this observation, cell-free supernatants from WT and Δ69700 strains were spotted onto lawns of P. aeruginosa indicator strains for R2 or F pyocins. Whereas supernatants from stationary WT cultures showed little inhibition (FIG. 1, Panel E) those from Δ69700 cultures showed substantial inhibition of both indicator strains (FIG. 1, Panel E). Collectively, these experiments confirm that deletion of 69700 results in elevated R/F pyocin production.

### Pyocin-Related Phenotypes in a 69700 Deletion are Mediated by a Deficit of XerC

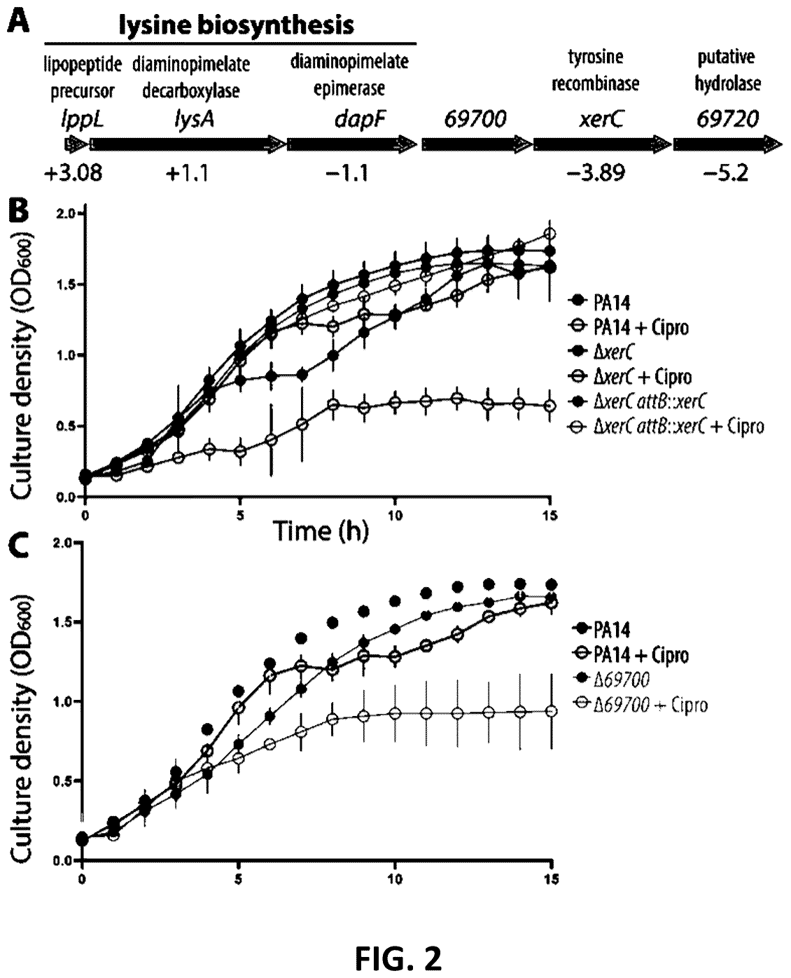

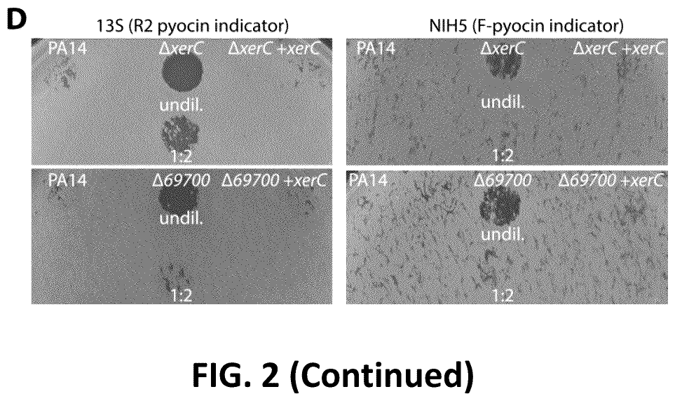

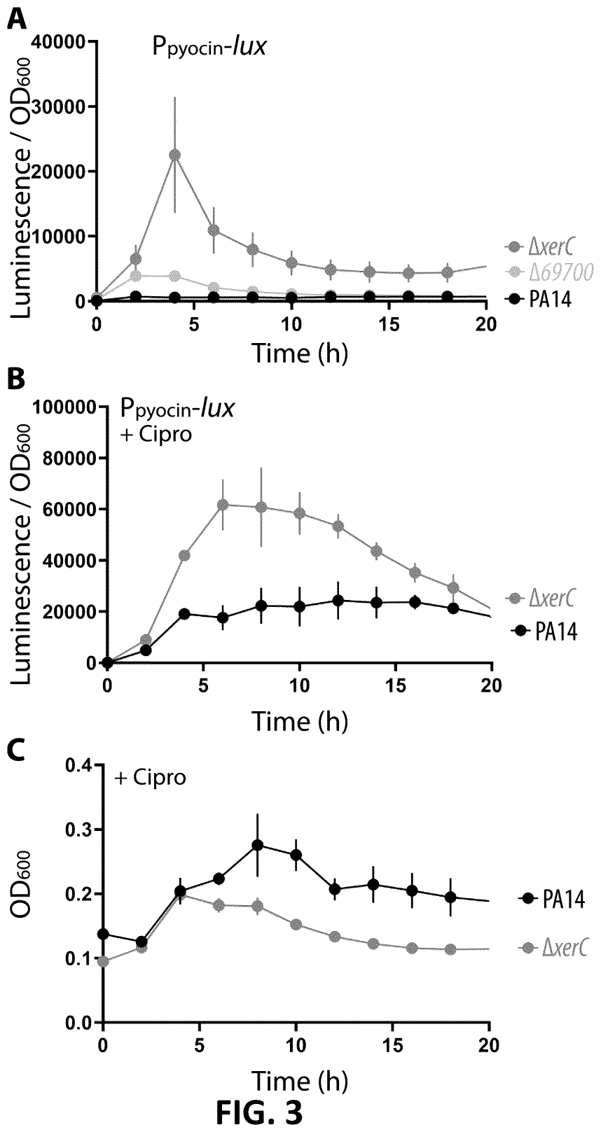

The transcriptomic data revealed that the two genes downstream of 69700 in its operon, xerC and 69720, were downregulated despite use of a markerless, in-frame deletion of 69700 (FIG. 2, Panel A). Since xerC (previously called sss) encodes a tyrosine recombinase enzyme, it was determined whether xerC contributed to elevated pyocin production. When a xerC deletion strain was challenged with ciprofloxacin it exhibited a sensitivity to ciprofloxacin similar to Δ69700 (FIG. 2, Panel B). Its sensitivity could be reversed by chromosomal complementation of xerC (driven by the operon promoter) at the attB locus (FIG. 2, Panel B). Importantly, the ciprofloxacin sensitivity of a Δ69700 strain was also reversed by chromosomal complementation with xerC (FIG. 2, Panel C), strongly indicating that decreased xerC expression is responsible for the ciprofloxacin-sensitivity phenotype of Δ69700 cells. The supernatants from both ΔxerC and Δ69700 strains exhibited significant clearing on the indicator strain, whereas no clearing was observed from either strain complemented with xerC (FIG. 2, Panel D). These experiments show that increased pyocin production in Δ69700 can be attributed to downregulation of xerC, so that a Δ69700 strain essentially acts as a hypomorphic allele of xerC. Therefore, ΔxerC strains were used for further characterization of the pyocin overproduction phenotype. Pyocin expression was quantified by chemiluminescence, placing a luciferase reporter under control of the R/F pyocin promoter. This system revealed that the xerC deletion increased R/F pyocin expression approximately five-fold greater than the 69700 deletion (FIG. 3, Panel A), consistent with the idea above that 69700 deletion resembles a hypomorphic xerC allele. Furthermore, treatment of ΔxerC cells with sublethal (0.03 μg/mL) ciprofloxacin substantially increased pyocin expression approximately threefold above treated WT cells (FIG. 3, Panel B). This result argues that xerC deficiency not only raises the basal level of pyocin expression but also magnifies antibiotic-induced pyocin production, which may explain the increased susceptibility of ΔxerC cells to ciprofloxacin (FIGS. 2, Panel B and FIG. 3, Panel C).

Pyocin Production in the xerC Deletion Requires PrtN but not RecA

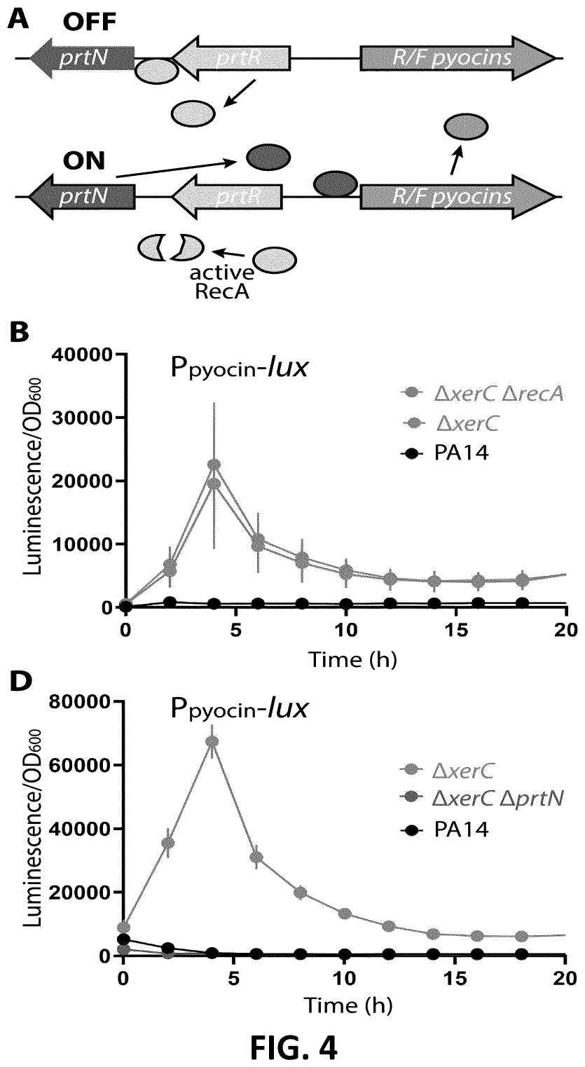

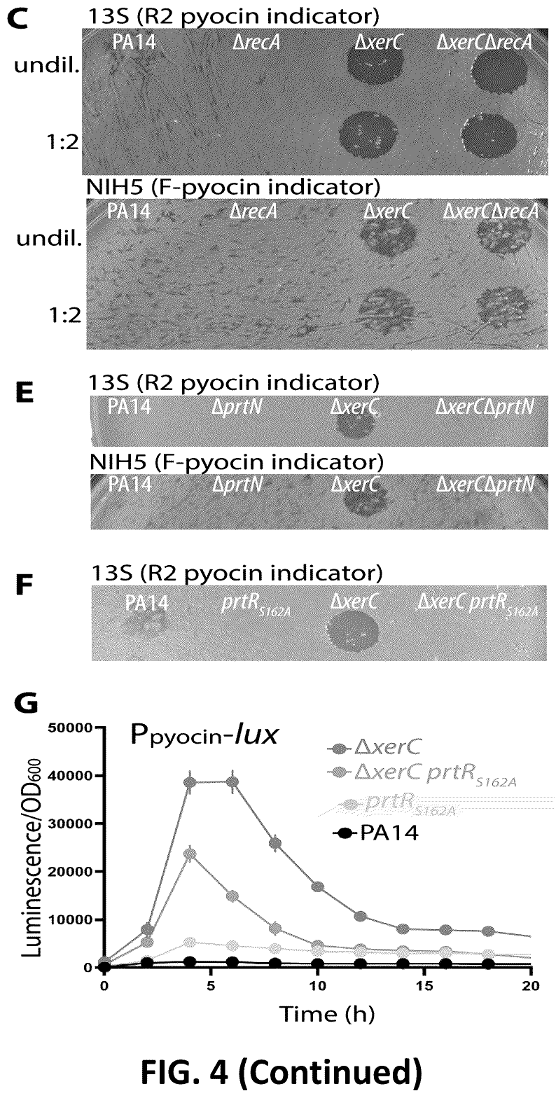

Next, the basis by which xerC deletion induces pyocin expression was explored. The known genetic mechanism for the expression of R-type pyocins requires the RecA protein in combination with a repressor, PrtR, and an activator, PrtN. Under uninduced conditions, PrtR binds to an operator in the prtN promoter and represses its expression (FIG. 4, Panel A). Activated RecA (i.e., when DNA damage is sensed) stimulates PrtR autocleavage, thereby derepressing prtN. PrtN then binds the promotor of the pyocin region at putative P-box regions, inducing expression of the R/F pyocin genes (FIG. 4, Panel A). Therefore, it was determined if pyocin expression was dependent on recA in the ΔxerC mutant. The expected low levels of pyocin expression were observed in WT cultures, whereas similar, highly elevated pyocin expression (via luciferase-stimulated chemiluminescence) (FIG. 4, Panel B) and killing activity (FIG. 4, Panel C), was observed for both ΔxerC and ΔxerC ΔrecA mutants. Collectively, these data show that production of pyocins in the absence of xerC occurs independently of RecA, thus bypassing a key element of the canonical regulatory mechanism for pyocin expression.

These results indicated that pyocin expression might also be independent of the pyocin gene activator PrtN, which was tested by deleting prtN in a ΔxerC background. Surprisingly, little to no pyocin expression or killing activity was observed in ΔxerC ΔprtN cultures (FIG. 4, Panels D and E, respectively), indicating that pyocin expression in ΔxerC strains requires PrtN. Then, it was determined whether PrtR autocleavage is required by introducing a gene encoding an uncleavable PrtR variant (S162A) at the native chromosomal locus. In both wild-type and ΔxerC strains, the uncleavable PrtR variant abolished detectable pyocin killing activity (FIG. 4, Panel F), whereas pyocin gene expression was diminished but not fully abolished (FIG. 4, Panel G). Thus, pyocin expression in xerC-deleted strains is strictly dependent on PrtN and at least partially repressible by PrtR. These results demonstrate the existence of a separate and previously unknown regulatory pathway for pyocins that bypasses RecA and is abetted by but not fully dependent on PrtR cleavage to relieve prtN repression.

### Pyocin Expression is Highly Heterogeneous Among Individual Cells

Elevated pyocin production, whether stimulated by DNA damage or induced by XerC deficiency, poses a challenge to cells. The holin and lysin enzymes encoded as part of the R/F pyocin cluster were substantially upregulated (approximately 8- and 16-fold, respectively) by 69700 deletion in the transcriptomic data. Upregulation of these lytic enzymes would presumably impose a fitness cost, as widespread cell lysis would severely hamper population growth. One means of managing costly phenotypes is through heterogeneity, wherein only some cells in a population engage in behaviors that are costly or deleterious but that benefit the remainder of the population. In support of this idea, two previous reports suggested that pyocins are heterogeneously expressed. First, in P. aeruginosa biofilms, a small subset of cells was observed to explosively lyse and release their DNA, and this lysis was dependent on the lysin encoded in the R/F pyocin gene cluster. Moreover, a GFP transcriptional reporter for the holin-encoding gene in the pyocin cluster was active in only a subset of cells. More recently, a study in P. protegens examined fluorescent fusions to tailocins, which are similar to pyocins, to directly observe intraspecies killing. In that work, tailocin production was likewise observed to be heterogeneous. Building on this evidence, it was thus hypothesized that pyocin expression would be heterogeneous, even when strongly induced by xerC deletion. This hypothesis was tested by visualizing pyocin expression at the single-cell level, constructing a GFP reporter for pyocin gene expression, and observing cells using fluorescence microscopy.

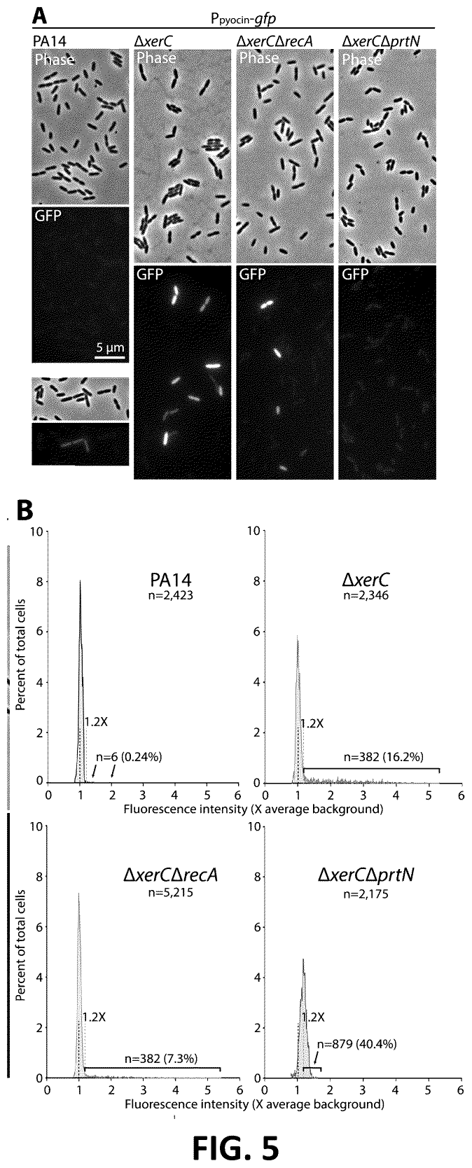

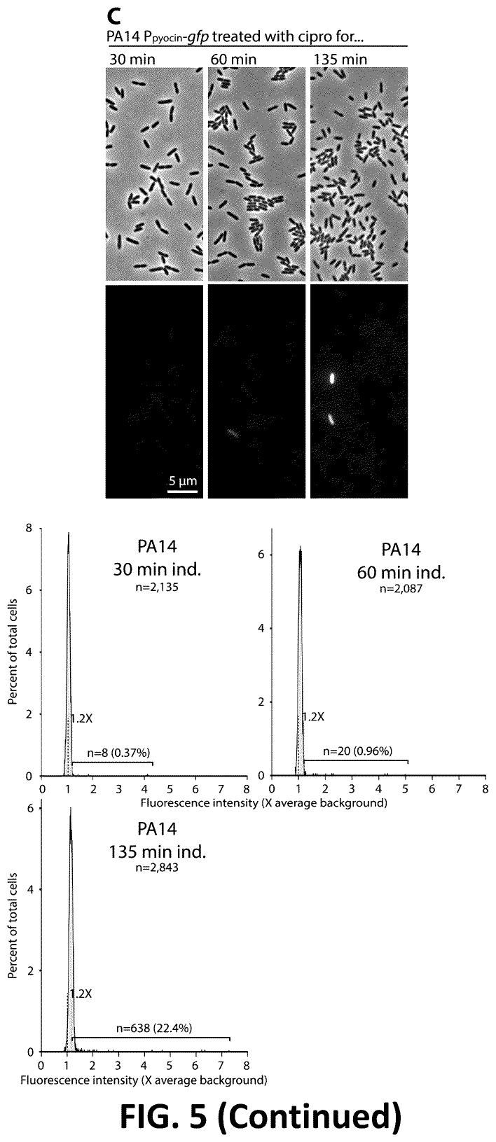

In wild-type cells, which showed very little pyocin gene expression in bulk assays, very few GFP-producing cells (0.24%) that were relatively dimly fluorescent were seen (FIG. 5, Panels A-B). In contrast, ΔxerC cells, while mostly GFP-negative, showed a much greater proportion of GFP-positive cells (16.2%) that varied in brightness but were generally much brighter than in the wild type. In a ΔxerC background, deletion of recA lowered the fraction of GFP-positive cells to 7.3%, consistent with the canonical RecA-PrtR pathway partially contributing to pyocin expression in ΔxerC cells. Deletion of prtN fully abolished the appearance of bright GFP-positive cells (FIG. 5, Panels A-B), in accord with the bulk data (FIG. 4, Panels B-D), but the overall fluorescence distribution was shifted so that some 40% of cells fell just above the cutoff for GFP positivity (FIG. 5, Panel B). Even in ΔxerC backgrounds, the majority of cells displayed little to no detectable GFP expression, supporting the hypothesis that a relatively small subset of cells strongly produces pyocins and is sacrificed for the fitness of the entire community.

The DNA damage-induced pathway was also examined using a 1 μg/ml concentration of ciprofloxacin that was previously used to induce SOS in P. aeruginosa. While more pyocin-expressing cells were observed in response to SOS induction, the proportion of bright GFP-expressing cells under the observed treatment durations were much lower than for ΔxerC cells (FIG. 5, Panel C), in accord with bulk assays (FIG. 3, Panels B-C). Interestingly, after 135 minutes of induction, a slight shift in overall fluorescence was observed, as in the ΔxerC ΔprtN strain, so that many cells fell at the bottom end of the GFP-positive range. Irrespective of the mode of induction, genetic or drug-induced, population heterogeneity appears to be a hallmark of pyocin gene expression, indicating the existence of a regulatory mechanism that introduces heterogeneity, even in an isogenic population, to limit sacrificial production of pyocins to only a few cells.

### Loss of XerC Recombinase Activity Modestly Increases Pyocin Expression

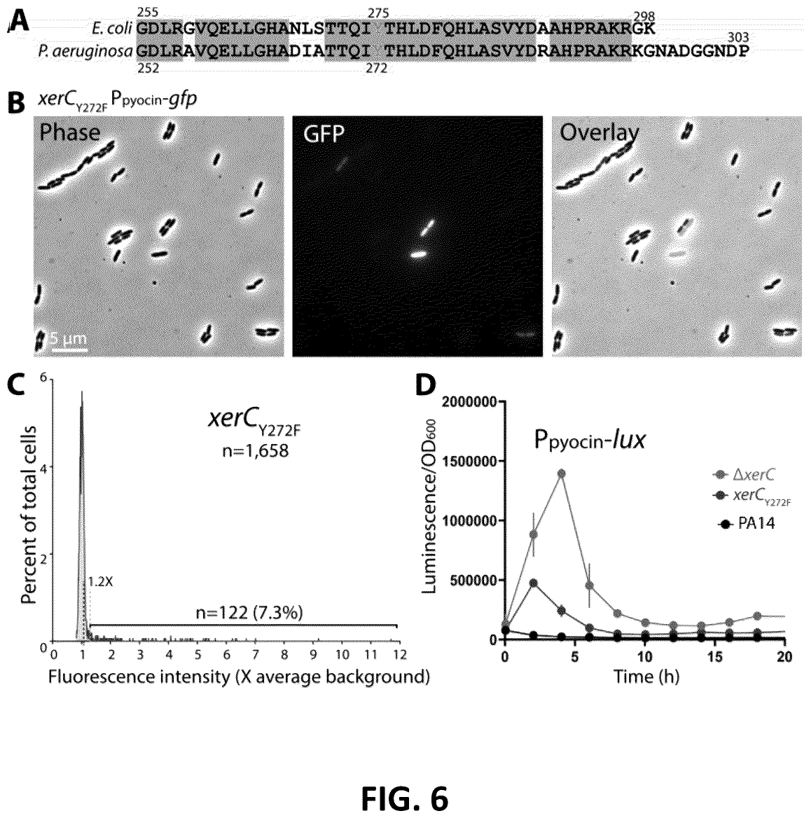

The increased levels of pyocin expression in ΔxerC strains, whether observed in bulk or at the single-cell level, made us ask whether it was specifically loss of XerC recombinase activity, or some other function of XerC, that induced pyocins. Thus, a recombinase-dead XerC_{Y272F} point mutant, in analogy to the catalytically inactive E. coli XerC_{Y275F} mutant, was constructed. The catalytic Tyr residue is located in a region of high sequence conservation between E. coli and P. aeruginosa (FIG. 6, Panel A). Qualitative microscopic observation of xerC_{Y272F} cells bearing a GFP reporter for pyocin expression (FIG. 6, Panel B) gave the impression that there were more pyocin-ON cells than in the wild-type, but not as many as in a xerC deletion. Quantitation of the fluorescence data (FIG. 6, Panel C) and bulk measurements with the pyocin luciferase reporter (FIG. 6, Panel D) indeed showed an intermediate phenotype for the point mutant when compared with wild-type or ΔxerC cells. These data argue that elevated pyocin expression in ΔxerC strains is not solely due to loss of XerC recombinase activity, implying another function of XerC with respect to pyocin expression.

### Pyocin Expression is a Terminal Phenotype

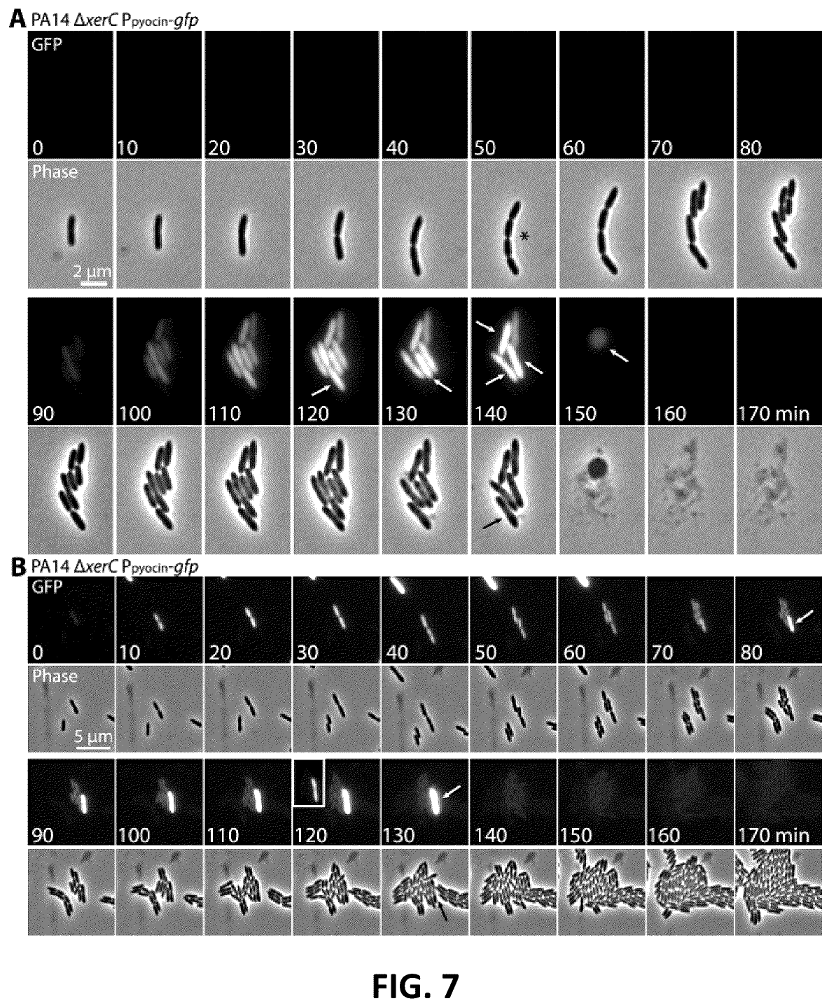

The heterogeneity of pyocin gene expression under every condition examined prompted expansion on the results and previous work by tracking the expression levels and fates of pyocin-producing cells using time-lapse fluorescence imaging. It was predicted that pyocin expression would result in cell death and/or lysis due to holin and lysin activity. A ΔxerC background was used because of the relatively frequent appearance of GFP-positive, pyocin-expressing cells in this strain (FIG. 5, Panel A). Time-lapse microscopy revealed several phenotypes. First, modest cell chaining was occasionally observed, with groups of 4-8 cells appearing to be incompletely separated after cell duplication, in the ΔxerC background (FIG. 7, Panel A, asterisk) that was short-lived and was dissimilar from the cellular filamentation or doublets reported for XerC-deficient E. coli. This demonstrates that loss of XerC has a less-severe effect on cell division and chromosomal partitioning in P. aeruginosa than in E. coli. Second, cells progressively increased in GFP reporter intensity until they would suddenly and explosively lyse, leaving behind visible debris (FIG. 7, Panel A). Third, spherical cells or spheroplasts was occasionally observed as an intermediate step in cell lysis (FIG. 7, Panel A, 150 min), consistent with previous observations. Fourth, it was frequently observed that a cluster of sister cells would all express the GFP reporter at about the same time (FIG. 7, Panel A), indicating that the initiating event for pyocin expression occurs a few cell divisions before GFP production can be detected. Fifth, a cell without strong GFP expression that nonetheless lysed was occasionally observed (FIG. 7, Panel A, 140 min, black arrow). Finally, pyocin production appeared to be a terminal phenotype, as nearly every cell observed expressing GFP later showed explosive lysis. Cells that appeared to turn off pyocin-GFP reporter expression after expressing some GFP were observed, albeit rarely (FIG. 7, Panel B), demonstrating that pyocin expression is reversible. However, in such cases, the peak GFP fluorescence was much lower than in cells that went on to lyse (compare the bright cell in FIG. 7, Panel B, 80 min and following, with the relatively dim surrounding cells). Collectively, these results indicate that a subset of cells commits to pyocin expression and then strongly expresses pyocins until lysis.

Disabling the Holin and Lysin Genes Delays but does not Prevent Cell Death

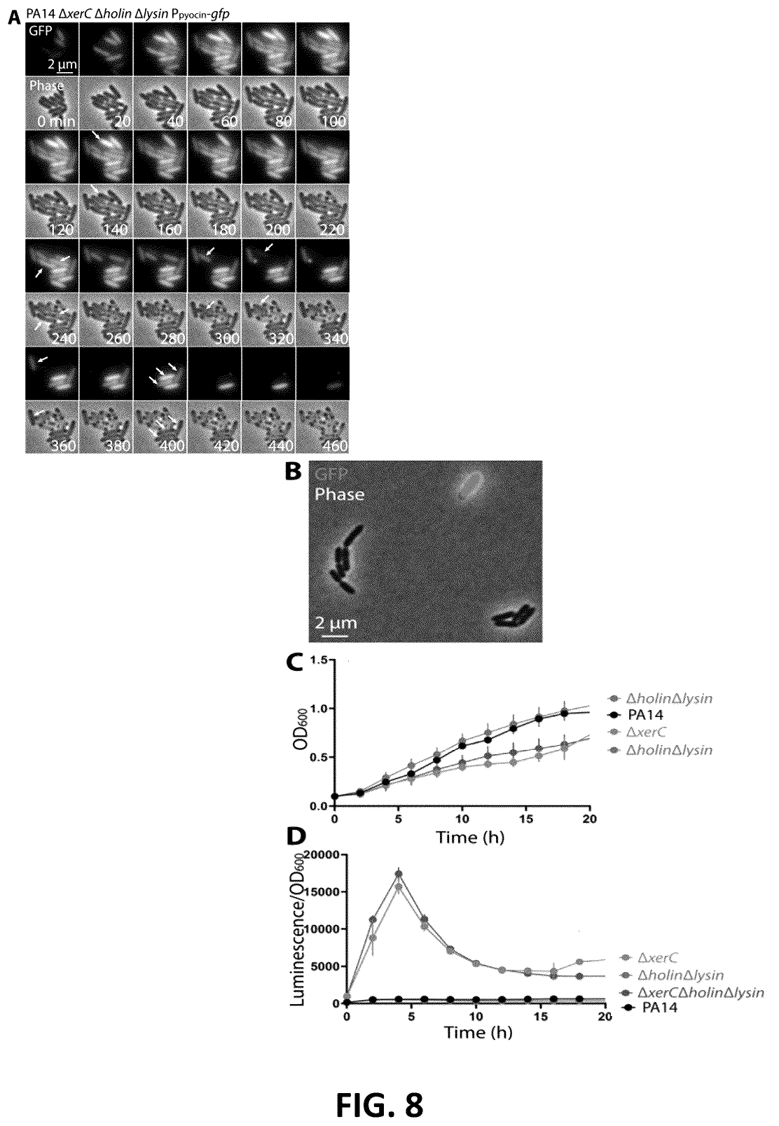

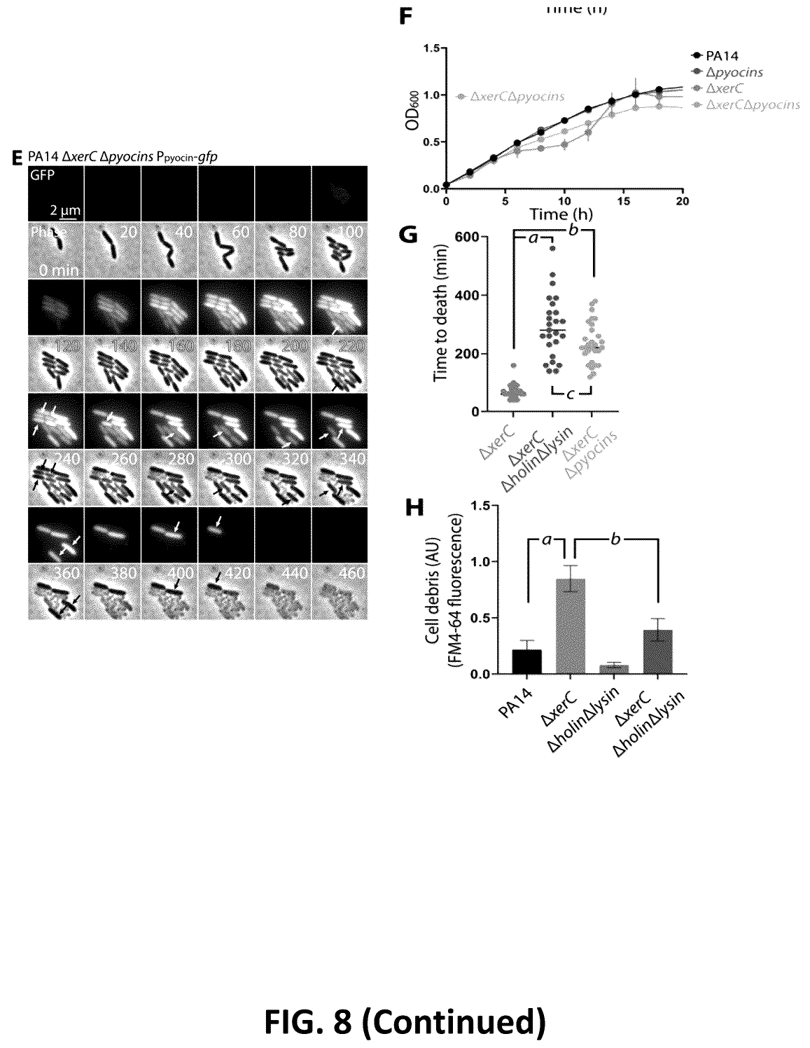

The striking explosive lysis of pyocin-producing cells was consistent with the long-standing model that holin and lysin enzymes are involved in pyocin release. To more formally test this model, the holin- and lysin-encoding genes were deleted from the pyocin gene cluster, and pyocin reporter cells were observed in bulk and microscopically. In ΔxerC cells deleted for holin and lysin, heterogeneous expression of GFP was observed that was qualitatively similar to the observations in the ΔxerC strain (FIG. 8, Panel B). In bulk, the ΔxerC Δholin Δlysin strain showed slightly faster growth (FIG. 8, Panel C) and greater luciferase expression (FIG. 8, Panel D) than a ΔxerC strain. Surprisingly, time-lapse imaging (FIG. 8, Panel A) revealed that pyocin-expressing cells still died, even in the absence of the holin and lysin, but with at least two key differences from strains encoding the holin and lysin. First, the time between detectable pyocin-GFP reporter expression and cell death in a strain lacking the holin and lysin genes was significantly delayed relative to a strain containing these genes (FIG. 8, Panel G). This longer delay between pyocin expression and cell death is a likely explanation for the faster cell growth and greater pyocin expression observed in bulk (FIG. 8, Panels C-D). Second, rather than explosively lysing, pyocin-expressing cells appeared to “deflate,” losing their GFP fluorescence and becoming brighter in phase-contrast images (FIG. 8, Panel A). In accord with this distinct mode of death, cells deleted for the holin and lysin also showed much less cell debris in culture supernatants than their holin- and lysin-replete counterparts (FIG. 8, Panel H).

### R/F Pyocin Production is not Fully Responsible for Cell Death of Pyocin-ON Cells

The non-explosive death of pyocin-ON holin-lysin mutant cells raised the question of whether other proteins encoded by the R/F pyocin gene cluster might be mediating cell death. Thus, a full deletion of the entire R/F pyocin gene cluster was constructed, and the fate of pyocin-ON cells (those activating the GFP transcriptional reporter under the control of the pyocin promoter) was tracked. Even with the full pyocin deletion, these cells nonetheless showed a death phenotype that mimicked that of the holin-lysin mutant (FIG. 8, Panel E), with slightly slower growth (FIG. 8, Panel F) and a similar time to death (FIG. 8, Panel G). This phenotype implies the existence of one or more factors that lead to cell death and are induced via the same pathway as R/F pyocins—perhaps under the control of PrtN.

### Discussion

For years, the only known inducer of pyocin expression, and even the expression of unrelated bacteriocins in other species, including E. coli, was DNA damage and the SOS response. In this Example, a new pathway for pyocin production in P. aeruginosa cells was identified that is both independent of and stronger than the SOS-induced pathway. This previously unknown pathway is induced by a lack of the XerC tyrosine recombinase. It is shown that pyocin production is strongly heterogeneous across a cell population, and that pyocin-ON cells display progressively strengthening expression before lysis or death. While not wishing to be bound by any particular theory, it is possible that the genetic circuitry underlying pyocin expression ensures that only some cells in a population commit to sacrificial cell lysis.

The more modest pyocin upregulation in a Δ69700 strain than in a ΔxerC deletion indicates that the alternative pathway for pyocin induction scales with the degree of XerC deficiency, with its complete absence causing a stronger phenotype than its downregulation. XerC has not been extensively studied in P. aeruginosa. The modest chaining phenotype observed in the ΔxerC deletion is consistent with a role in chromosome separation, but a less critical role than in Escherichia coli. While it is not yet fully understood how the absence of XerC causes pyocin gene upregulation, but its RecA independence, and the lack of SOS gene upregulation in a Δ69700 deletion, argue against DNA damage or SOS responses as an initiating factor. Still, at least some contribution of RecA-dependent processes to pyocin expression in ΔxerC cells cannot be ruled out, as recA deletion reduced the proportion of GFP-positive cells in the analyses (although it did not substantially change observable pyocin expression in bulk). A partial dependence on RecA would be consistent with previous E. coli work showing at least some SOS-induced cells in a ΔxerC background.

The less-severe phenotype of the XerC_{Y272F} recombinase-dead mutant compared to the full deletion indicates that XerC may have multiple functions in regulating pyocin expression. It will be interesting to learn whether deletion of other recombinases, such as XerD, similarly elicits pyocin production. The finding that pyocin upregulation in ΔxerC can occur independently of RecA, is partially repressed by PrtR, and is dependent on PrtN demonstrates that PrtR cleavage and prtN derepression can occur by a previously unknown mechanism that is independent of the SOS response. Previous work has shown that certain other mutations, such as in the oligoribonuclease-encoding orn gene, upregulate pyocin expression but in a RecA/SOS-dependent manner. More recently, inactivation of fis (factor for inversion stimulation) increased pyocin production by increasing prtN expression by approximately 2.7-fold; Fis was shown to directly bind to the prtN promoter as an apparent repressor. This effect appeared to be independent of the SOS response but was substantially weaker than the impact of reduced XerC levels, as prtN was upregulated approximately 10-fold, even in the Δ69700 strain used for the transcriptomic studies. Hence, the phenotype of ΔxerC is distinguished both by its SOS independence and by its strong prtN and pyocin expression.

The time-lapse microscopy showed that pyocin expression is rarely “turned off” once activated, with the vast majority of pyocin-positive cells growing brighter and brighter until cell lysis or death. Interestingly, strong heterogeneity was always observed, with individual cells typically being strongly on or completely off. It was hypothesized that positive auto-feedback in prtN expression may be involved in this pattern, as it would be one straightforward mechanism to explain the observed heterogeneity. It was noted that heterogeneous responses of individual bacterial cells to a particular internal or environmental condition are common, with different underlying mechanisms.

Initially, it was surprising to find that pyocin-producing cells died even in the absence of the holin and lysin or in the absence of the entire R/F pyocin gene cluster. Although these cells do not explosively lyse, the loss of GFP fluorescence indicates that the integrity of the cell envelope is compromised. What kills these cells? It is tempting to speculate that one or more other lytic proteins are also under the control of PrtN. The AlpBCDE proteins, which include phage holin-like enzymes that can cause cell lysis, are strong candidates, as alpB was upregulated approximately 6-fold in the Δ69700 transcriptomic data. Activation by PrtN of the alpBCDE cluster, typically activated by its own activator, AlpA, would be particularly interesting, with such activator crosstalk perhaps serving as a fail-safe to ensure cell lysis when pyocins are expressed. Another possibility is that the absence of XerC may independently induce the Alp system, in accord with the observation of occasional cell lysis without strong pyocin expression (FIG. 7, Panel A). Another lytic protein, CidA, was downregulated approximately 2-fold in the transcriptomic data, and so it is considered a less likely (but not by way of limitation) candidate.

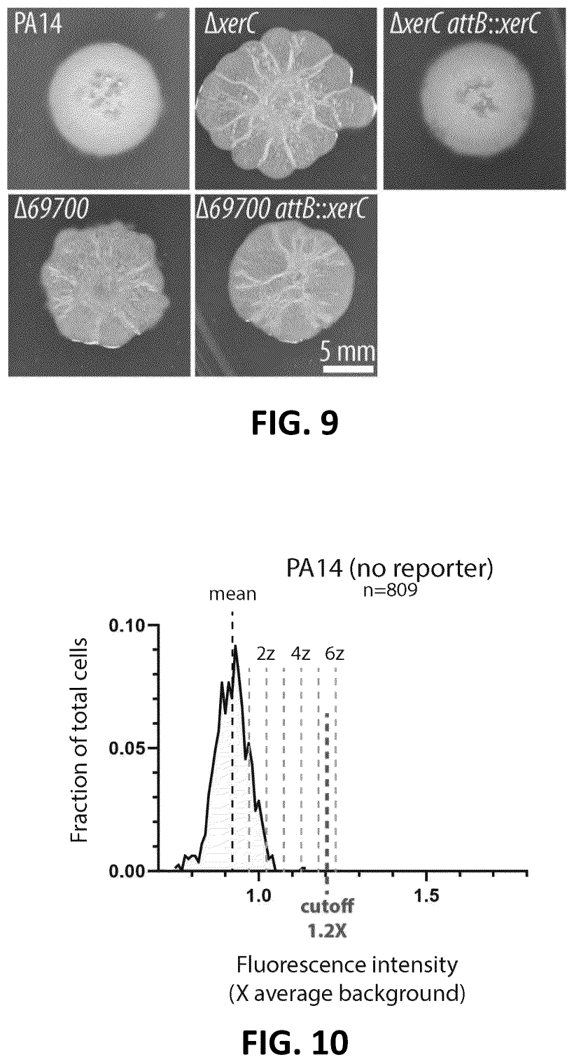

It was briefly noted that the experiments identifying xerC as the critical gene for the pyocin phenotype also hinted at a potential role for xerC in biofilm formation, at least as assessed by colony morphology (FIG. 9). The failure of xerC complementation to reverse the wrinkled morphology of a Δ69700 strain (FIG. 9) demonstrates that 69700 is itself a determinant of biofilm formation, independent of the effects of 69700 deletion on xerC expression.

Finally, the results showed a striking effect of ciprofloxacin treatment on ΔxerC cells, with elevated pyocin expression (FIG. 3, Panel C) and, accordingly, poorer growth. This finding indicates that anti-pseudomonal agents identified by the methods disclosed herein (including, for example but not by way of limitation, chemical inhibitors of XerC) might sensitize P. aeruginosa to fluoroquinolone antibiotics via increased production of pyocins. Notably, recent work indicates that many clinical isolates are sensitive to pyocins and/or produce pyocins themselves. Hence, the present disclosure envisions combination treatment with an anti-pseudomonal agent identified by the methods disclosed herein (such as, but not limited to, a pyocin-stimulating XerC inhibitor) plus a fluoroquinolone. P. aeruginosa cells would not only become more antibiotic sensitive but also release increased quantities of P. aeruginosa-killing pyocins as they die, amplifying the bactericidal effect.

### Materials and Methods

### Strains and Growth Conditions

Escherichia coli SM10 and Pseudomonas aeruginosa PA14 were grown in Luria-Bertani (LB) Lennox broth (10 g/L tryptone, 5 g/L yeast extract, 5 g/L NaCl) or on LB agar plates fortified with 1.5% Bacto agar at 37° C. When appropriate, 25 μg/ml irgasan (to specifically select for P. aeruginosa) plus 75 μg/ml tetracycline, 25 μg/ml irgasan plus 75 μg/ml gentamicin, 25 μg/ml tetracycline, or 20 μg/ml gentamicin was added to liquid or solid media. P. aeruginosa was also selected over E. coli for some strains by growth on VBMM containing citrate as the sole carbon source. The strains used in this work are listed in Tables 1-3. Markerless deletions were generated using the pEXG2 vector with counterselection on LB plates containing 6% sucrose or no-salt LB plates containing 15% sucrose and were screened by colony PCR for the presence of deletions. Complementation and reporter strains were constructed by integration of the mini-CTX-1 vector at the neutral chromosomal attB locus. Modes of strain and plasmid construction are given in the Supplemental Materials.

### Biofilm Assays

P. aeruginosa biofilm studies were conducted using on solid (1% agar) M6301 medium composed of 100 M KH₂PO₄, 15.14 mM (NH₄)₂SO₄, and 0.36 μM FeSO₄·H₂O (pH-balanced to 7.0 using 10 M KOH) (Cabeen, 2014); after autoclaving and before use, 0.5% glycerol, 1 mM MgSO₄, and 0.2% casamino acids (BD Bacto, USA) were added. Plates containing 40 ml of M6301 with 1% agar were poured fresh for each experiment and allowed to harden for 6-7 hours. P. aeruginosa cultures grown at 37° C. for 6-8 h in 3 ml LB were back-diluted to an OD₆₀₀ of 1.0, and 2 μl of the diluted cultures were spotted on M6301 agar plates. The plates were incubated right-side-up at 25° C. and were typically photographed after 4 or 6 days, as indicated.

### RNA Isolation and Sequencing

RNA sequencing was achieved by growing the strain of interest in quadruplicate on solid M6301-1% agar plates for 3 days. Total RNA was isolated from homogenized colonies using the New England Biolabs Monarch Total RNA Miniprep Kit. Subsequent quality-control steps, the ribosomal RNA depletion, Illumina library preparation, and paired-end high-throughput Illumina sequencing were performed by Novogene (Beijing, China). Sequence mapping and analysis were performed at the Oklahoma University Health Sciences Center Laboratory for Molecular Biology and Cytometry Research using CLC software.

### Growth Curve Analysis

Strains of interest were grown on LB plates overnight, then inoculated into LB liquid broth until late stationary phase was reached (about 18 hours). Strains were then diluted 1,000-fold into fresh medium and grown to early exponential phase (about 4 hours). The optical density at 600 nm (OD₆₀₀) was measured, and all cultures were normalized to an OD₆₀₀ equal to 0.1. A 96 well plate containing 160 μL of LB liquid broth containing 0.03 μg/mL ciprofloxacin was inoculated with 20 μL of the normalized cultures. The plate was incubated in a BioTek Synergy H1 plate reader (BioTek, USA) at 37° C. for 20 h with orbital shaking. OD₆₀₀ measurements were obtained every 2 minutes.

### Pyocin Indicator Assays

Strains of interest were grown in 10 mL of LB liquid broth at 37° C. until stationary phase was reached (about 12 hours). OD₆₀₀ measurements were obtained, and the cultures were normalized to the lowest value with a volume of 8 mL in 15 mL centrifuge tubes. The cells were then pelleted by centrifugation (4,500 g, 10 min, 25° C.); the supernatants were harvested and filtered using a 0.22-μm syringe filter to remove any remaining cells. Filtered supernatants were stored at 4° C. and used within 2-3 d. Indicator strains were grown in 3 mL of LB liquid at the same time as the strains of interest. The dense cultures were diluted 1,000-fold into a microcentrifuge tube, then 150 μL of the diluted cultures were spread plated onto a LB plate using sterile glass beads. The filtered supernatants of the strains of interest were then used undiluted or diluted with sterile LB. Ten microliters of undiluted or diluted supernatants were spotted on top of the indicator strain plates. The plates were then incubated at 37° C. overnight.

### Kinetic Luciferase Assay

Strains of interest were cultured as described in the growth curve analysis section. Luminescence was measured in black or white clear-bottom 96-well microtiter plates at 3-min intervals at a sensitivity (gain) setting of 135 or 200 together with the OD₆₀₀ for 20 hours on a BioTek Synergy H1 plate reader (BioTek, USA). Final luciferase activity values were calculated by normalizing luciferase luminescence to culture density. Because of differences in the plates (black or white) and gain settings, the luminescence/OD values are not always comparable from graph to graph.

### Fluorescence Microscopy

Strains of interest were grown in 3 mL of LB liquid broth overnight to obtain a saturated culture. They were then diluted 1,000-fold in fresh LB and grown to early exponential phase (about 4 hours). Cultures were concentrated by centrifuging 1 mL of exponential culture into a microcentrifuge tube at 5000 g for 1 min and resuspending in 100 μL. Cells were immobilized by spotting 0.5 μL of the concentrated mixture onto the pad and covering with cover glass. Imaging was performed on a Nikon Eclipse Ti inverted fluorescence microscope equipped with a Photometrics Prime 95B sCMOS digital camera, a Lumencor SOLA SE II 365 LED Light Engine, and an OKO temperature-controlled enclosure. Snapshot images of the slides were taken at 100× magnification in both phase and GFP channels. Automated timelapse imaging was performed at 37° C. For quantification of GFP-positive cells, images were analyzed using the MicrobeJ plugin for ImageJ, segmenting on phase-contrast and taking the mean GFP values of the corresponding fluorescence images. Segmentation was performed with default values except that a minimum and maximum areas of 100 and 400 px were used, and circularity was delimited from 0-0.9. For options, “exclude on edges,” “shape descriptors,” “segmentation,” and “intensity” were selected. A threshold of 1.2 times the average background fluorescence was selected to denote GFP positivity, as 100% of PA14 cells without a GFP reporter fell below this threshold, which was approximately 5.5 standard deviations above the mean fluorescence of reporter-free cells (FIG. 10).

### Quantification of Cell Debris/Vesiculation

Strains of interest were grown in 10 mL of LB broth at 37° C. for 12 hours. Once grown, the cultures were equalized to the culture with the lowest OD₆₀₀. The cells were then pelleted by centrifugation (4500 g, 12 min, 4° C.); the supernatants were harvested and filtered using a 0.22-μm syringe filter to remove excess bacteria. The filtrates were then centrifuged again in a TLA100.3 rotor at 80,000 rpm for 30 minutes at 4° C. in a Beckman Coulter TL-100 ultracentrifuge. The supernatants were gently decanted, and the pellets were resuspended in 100 μL PBS supplemented with 0.2 M NaCl. The resuspended material was stained using FM4-64 at a final concentration of 3.3 μg/mL for 10 min at 37° C. Fluorescence was measured using a BioTek Synergy H1 plate reader (BioTek, USA) with an excitation wavelength of 506 nm and an emission wavelength of 700 nm.

TABLE 1
Previously Known Pseudomonas aeruginosa
strains used in the Examples

| Strain | Genotype or description | Source or reference |
|---|---|---|
| PA14 | Laboratory wild-type strain | Laboratory stock; Stephen |
| (MTC1) | of P. aeruginosa | Lory, Harvard Medical School |
| MTC590 | PA14 ΔamrZ | Cabeen et al., 2016 (Mol |
| | | Microbiol, 99: 557-70) |
| MTC1398 | PA14 ΔamrZ Δ69700 | Cabeen et al., 2016 |
| MTC1513 | PA14 Δ69700 | Cabeen et al., 2016 |
| MTC2191 | S13 | Pradeep Singh Lab |
| | | (University of Washington) |
| MTC2192 | PML1516d | Pradeep Singh Lab |
| MTC2193 | NIH5 | Pradeep Singh Lab |

TABLE 2
Newly Constructed Pseudomonas aeruginosa strains

| Strain | Genotype or description |
|---|---|
| PA14 | Laboratory wild-type strain of P. aeruginosa |
| (MTC1) | |
| MTC590 | PA14 ΔamrZ |
| MTC1398 | PA14 ΔamrZ Δ69700 |
| MTC1513 | PA14 Δ69700 |
| MTC2191 | S13 |
| MTC2192 | PML1516d |
| MTC2193 | NIH5 |
| MTC2252 | PA14 ΔxerC attB::CTX-1-P₀₇₉₉₀-gfp, Tet^{R} |
| MTC2262 | PA14 ΔxerC attB::CTX-1-P_{lppL}-xerC, Tet^{R} |
| MTC2264 | PA14 Δ69700 attB::CTX-1-P_{lppL}-xerC, Tet^{R} |
| MTC2266 | PA14 ΔxerC |
| MTC2274 | PA14 ΔrecA |
| MTC2276 | PA14 ΔprtN |
| MTC2277 | PA14 attB::CTX-1-P₀₇₉₉₀-gfp, Tet^{R} |
| MTC2280 | PA14 attB::CTX-1-P₀₇₉₉₀-lux, Tet^{R} |
| MTC2281 | PA14 Δ69700 attB::CTX-1-P₀₇₉₉₀-lux, Tet^{R} |
| MTC2284 | PA14 Δholin Δlysin attB::CTX-1-P₀₇₉₉₀-lux, Tet^{R} |
| MTC2285 | PA14 Δ69700 Δholin Δlysin attB::CTX-1-P₀₇₉₉₀-lux, Tet^{R} |
| MTC2288 | PA14 ΔxerC ΔrecA |
| MTC2289 | PA14 ΔxerC AprtN |
| MTC2291 | PA14 ΔxerC ΔrecA attB::CTX-1-P₀₇₉₉₀-gfp, Tet^{R} |
| MTC2292 | PA14 ΔxerC AprtN attB::CTX-1-P₀₇₉₉₀-gfp, Tet^{R} |
| MTC2293 | PA14 ΔxerC Δholin Δlysin attB::CTX-1-P₀₇₉₉₀-gfp, Tet^{R} |
| MTC2294 | PA14 ΔxerC Δholin Δlysin |
| MTC2295 | PA14 Δholin Δlysin |
| MTC2297 | PA14 ΔxerC attB::CTX-1-P₀₇₉₉₀-lux, Tet^{R} |
| MTC2298 | PA14 ΔxerC ΔprtN attB:CTX-1-P₀₇₉₉₀-lux, Tet^{R} |
| MTC2299 | PA14 ΔxerC Δholin Δlysin attB::CTX-1-P₀₇₉₉₀-lux, Tet^{R} |
| MTC2301 | PA14 ΔxerC ΔrecA attB::CTX-1-P₀₇₉₉₀-lux, Tet^{R} |
| MTC2302 | PA14 ΔrecA attB::CTX-1-P₀₇₉₉₀-lux, Tet^{R} |
| MTC2303 | PA14 ΔprtN attB::CTX-1-P₀₇₉₉₀-lux |
| MTC2304 | PA14 ΔxerC prtR_{S162A} |
| MTC2305 | PA14 prtR_{S162A} |
| MTC2307 | PA14 ΔxerC prtR_{S162A} attB::CTX-1-P₀₇₉₉₀-lux, Tet^{R} |
| MTC2308 | PA14 prtR_{S162A} attB::CTX-1-P₀₇₉₉₀-lux, Tet^{R} |
| MTC2324 | PA14 ΔxerC Δpyocins (Δ07990-08300) |
| MTC2326 | PA14 Δpyocins (Δ07990-08300) |
| MTC2332 | PA14 ΔxerC Δpyocins attB::CTX-1-P₀₇₉₉₀-gfp, Tet^{R} |
| MTC2337 | PA14 xerC_{Y272F} |
| MTC2339 | PA14 xerC_{Y272F} attB::CTX-1-P₀₇₉₉₀-lux, Tet^{R} |
| MTC2341 | PA14 xerC_{Y272F} attB::CTX-1-P₀₇₉₉₀-gfp, Tet^{R} |
| MTC2441 | PA14 xerC_{Y272F} ΔrecA |
| MTC2444 | PA14 xerC_{Y272F} ΔrecA attB::CTX-1-P₀₇₉₉₀-lux |
| MTC2448 | PA14 ΔrecA attB::CTX-1-P₀₇₉₉₀-gfp |

TABLE 3
Escherichia coli strains used in the Examples

| Strain | Genotype or description | Source or reference |
|---|---|---|
| MTC27 | SM10 (F- endA1 hsdR17 | Simon et al. (1983) |
| | supE44 thi-1 λ- recA1 gyrA96 | Bio/Technology, |
| | relA1); E. coli mating strain for | 1: 784-791. |
| | conjugation with P. aeruginosa | |
| MTC570 | SM10 pEXG2-ΔamrZ, Gent^{R} | Cabeen et al., 2016 |
| MTC1346 | SM10 pEXG2-Δ69700, Gent^{R} | Cabeen et al., 2016 |
| MTC2163 | SM10 pEXG2-Δ07990-08300 | New |
| | (Δpyocin) | |
| MTC2176 | SM10 pEXG2-ΔprtN, Gent^{R} | New |
| MTC2179 | SM10 pEXG2-ΔrecA, Gent^{R} | New |
| MTC2212 | SM10 pEXG2-ΔxerC, Gent^{R} | New |
| MTC2220 | SM10 pEXG2-Δlysin, Gent^{R} | New |
| MTC2226 | SM10 pEXG2-Δholin, Gent^{R} | New |
| MTC2261 | SM10 pCTX-1-P_{lppL}-xerC, Tet^{R} | New |
| MTC2286 | SM10 pCTX-1-P₀₇₉₉₀-gfp, Tet^{R} | New |
| MTC2287 | SM10 pCTX-1-P₀₇₉₉₀-lux, Tet^{R} | New |
| MTC2306 | SM10 pEXG2-prtR_{S162A}, Gent^{R} | New |
| MTC2334 | SM10 pEXG2-xerC_{Y272F} | New |

### Example 2

In this Example, four questions about the pathways by which DNA-damaging drugs stimulate pyocin expression and their relationship to XerC recombinase activity were addressed. The first query is whether DNA damage-mediated induction of pyocin expression in ΔxerC cells is mediated by the canonical RecA-dependent pathway and whether the pyocin expression activator PrtN is required. Next, it was examined whether the very high level of pyocin expression in drug-treated cells remains heterogeneous at the single-cell level. The next query was whether pyocin induction in strains inactivated for XerC recombinase activity occurs independently of RecA, as in ΔxerC strains. The final query of this Example is how a previously characterized antibacterial Holliday junction-binding, tyrosine recombinase-inhibiting hexapeptide impacts P. aeruginosa cell growth and/or pyocin expression. The findings reveal that, surprisingly, DNA damage-induced pyocin expression in ΔxerC cells is independent of RecA but remains dependent on PrtN. Even strongly expressing cell populations maintain their heterogeneity across individual cells. In sharp contrast to xerC deletion, XerC enzymatic inactivation appears to exclusively stimulate the canonical, RecA-dependent pyocin activation pathway, indicating a second, recombination-independent function for XerC in pyocin regulation. Finally, the hexapeptide tyrosine recombinase inhibitor can impede growth of P. aeruginosa but does not stimulate pyocin expression, indicating that its primary mode of growth inhibition does not resemble XerC genetic inactivation or deletion. The entire contents of Bronson et al. (Microbiol. Spectr. (2022 Aug. 31) 10(4):e0116722) are hereby expressly incorporated herein by reference in this Example.

### Results

RecA-Independent Stimulation of Pyocin Expression by Ciprofloxacin in a xerC Deletion Background

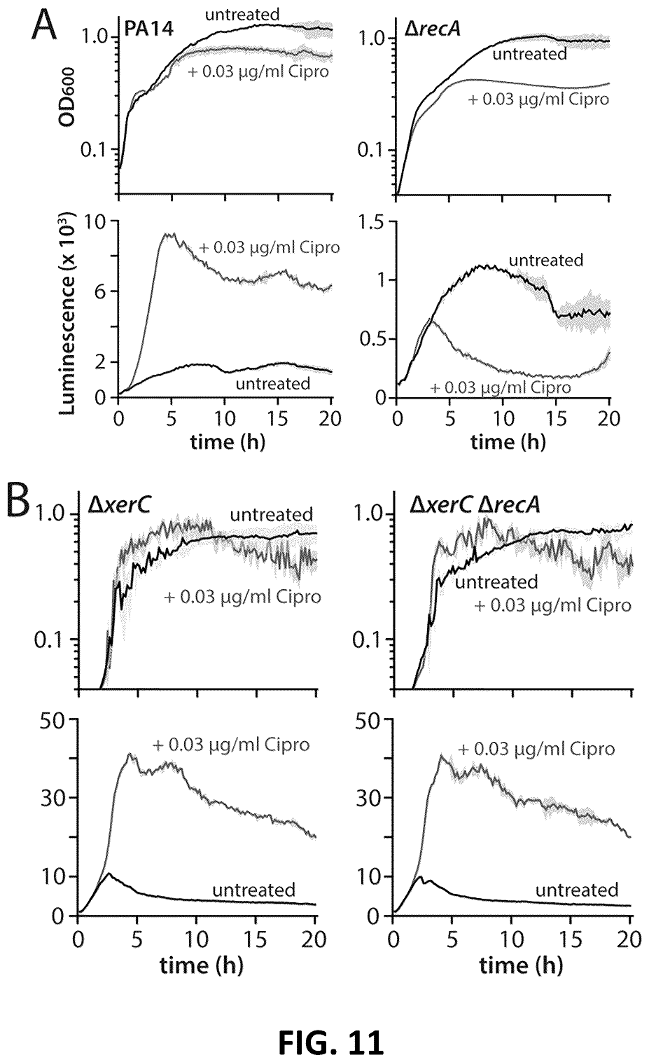

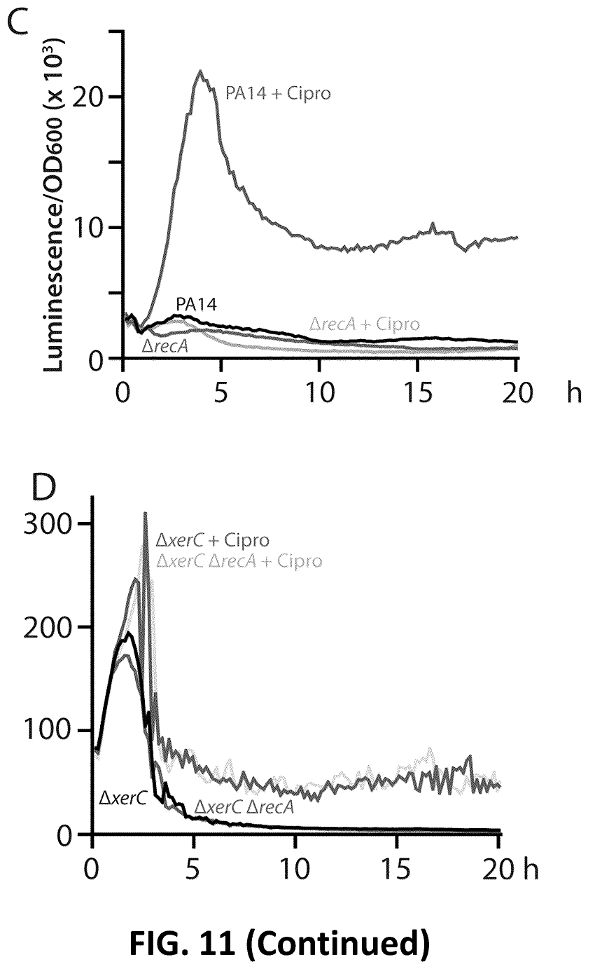

Ciprofloxacin is a known inducer of pyocin expression via the canonical RecA-dependent pathway for pyocin expression. In this pathway, ciprofloxacin treatment blocks gyrase activity and causes DNA damage, thereby activating RecA; active RecA stimulates cleavage of PrtR, a repressor of prtN. The resulting derepression of prtN, which encodes an activator of pyocin gene expression, causes elevated pyocin production. First, the RecA dependence of ciprofloxacin-induced pyocin expression was confirmed in wild-type cells bearing a luminescent reporter for R/F pyocin expression. Treatment with a sublethal concentration of ciprofloxacin (0.03 μg/ml) strongly stimulated pyocin expression, and deletion of recA abolished ciprofloxacin-stimulated pyocin expression (FIG. 11, Panels A, C; note the different Y-axis scales for luminescence graphs), consistent with the canonical model. Example 1 reported that a ΔxerC strain not only displayed markedly elevated pyocin expression relative to the wild type, but also showed a substantial further increase in pyocin expression upon ciprofloxacin treatment. As the elevated basal expression of pyocins in ΔxerC strains is independent of the canonical RecA-mediated pathway, it was reasoned that the further increase in pyocin expression stimulated by ciprofloxacin in ΔxerC cells might be due to simultaneous activation of the canonical, RecA-dependent pathway. To test this notion, ΔxerC βrecA cells were treated with ciprofloxacin, and pyocin expression was examined. Ciprofloxacin-stimulated pyocin expression in ΔxerC cells occurred irrespective of the presence of RecA, with indistinguishable phenotypes (FIG. 11, Panels B, D). This result indicated that the further elevation of pyocin expression under ciprofloxacin treatment in a ΔxerC background is not due to additional activation of the canonical, RecA-dependent pathway. Instead, the ciprofloxacin-mediated increase of pyocin expression in ΔxerC cells occurs independently of RecA.

RecA-Independent Stimulation of Pyocin Expression in ΔxerC Cells by Mitomycin C

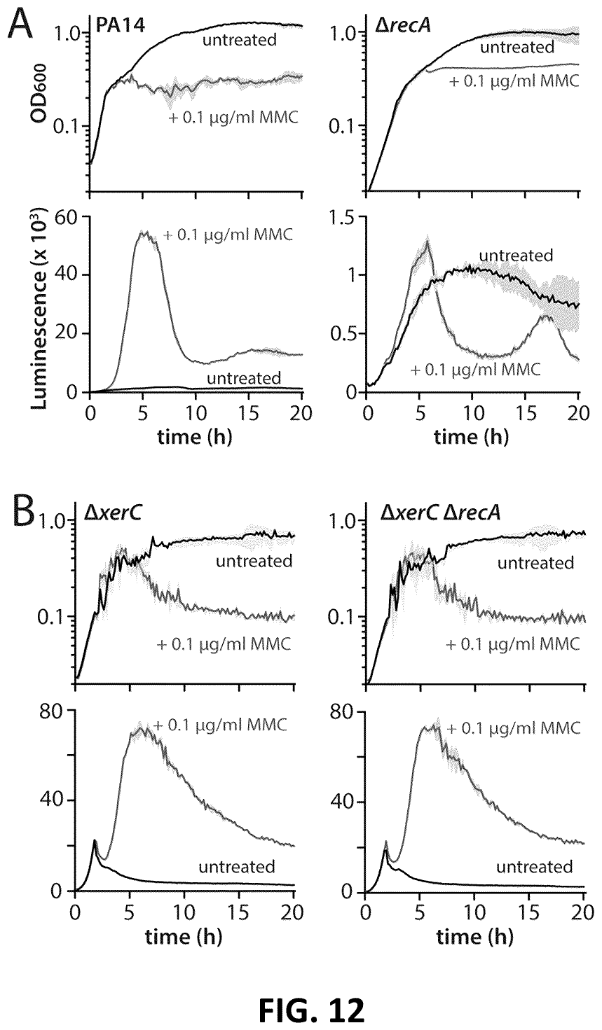

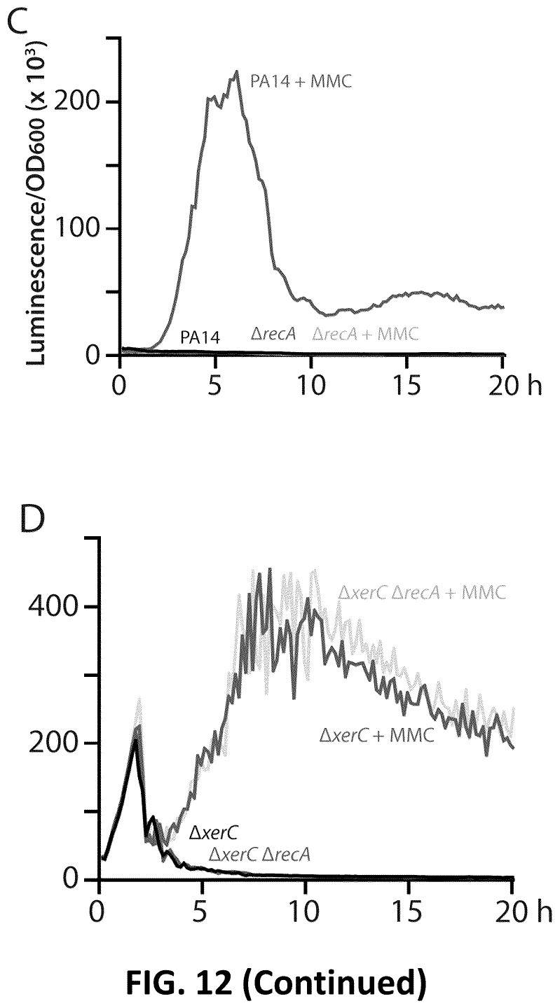

It was next asked whether treatment with mitomycin C (MMC) would show the same pattern of RecA dependence as ciprofloxacin. MMC differs from ciprofloxacin as it directly damages DNA (it is a DNA crosslinking agent), and it is a known strong activator of pyocin expression in P. aeruginosa. When wild-type cells were treated with 0.1 μg/ml MMC, which inhibited cell growth to a slightly greater degree than 0.03 μg/ml ciprofloxacin (FIGS. 11, Panel A, and FIG. 12, Panel A), strong pyocin expression was observed that peaked at approximately tenfold that induced by ciprofloxacin on an OD-normalized basis (FIG. 11, Panels A, C; FIG. 12, Panels A, C; note the different Y-axis scales for luminescence graphs). As with ciprofloxacin, deletion of recA fully abolished this very strong MMC-stimulated pyocin induction. MMC treatment also strongly increased pyocin expression in ΔxerC cells, with a later, but approximately twofold stronger, peak of expression than in untreated cells (FIG. 12, Panels B, D). This MMC-mediated increase, as with ciprofloxacin, was independent of RecA (FIG. 12, Panels B, D). Collectively, these results indicate that in ΔxerC cells, pyocin expression occurs via a RecA-independent pathway that, surprisingly, is strongly inducible by DNA-damaging agents.

### The RecA-Dependent and RecA-Independent Pathways for Pyocin Expression Both Require PrtN

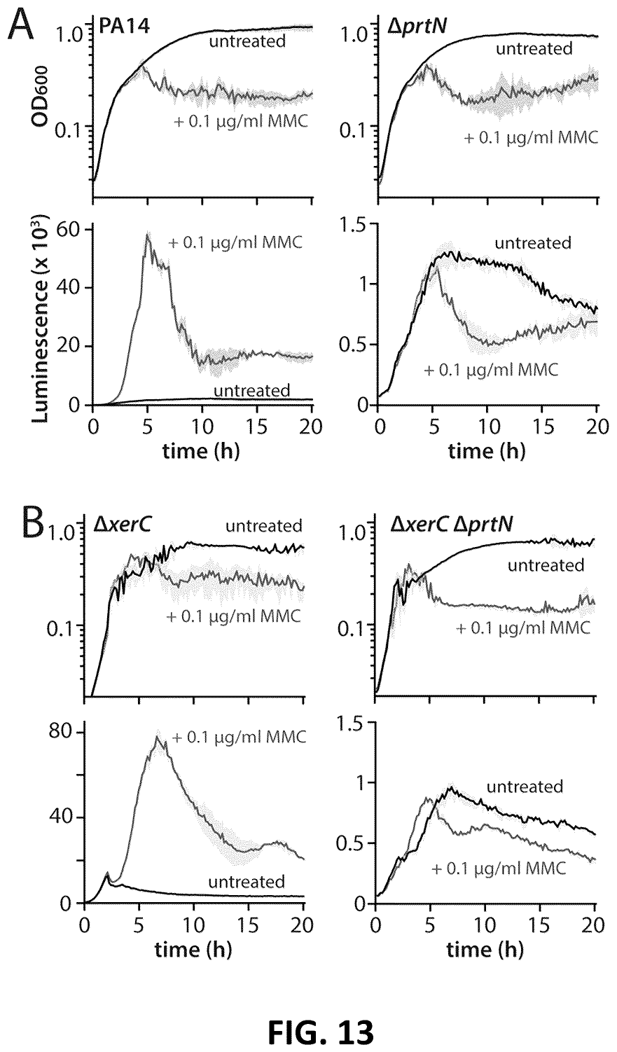

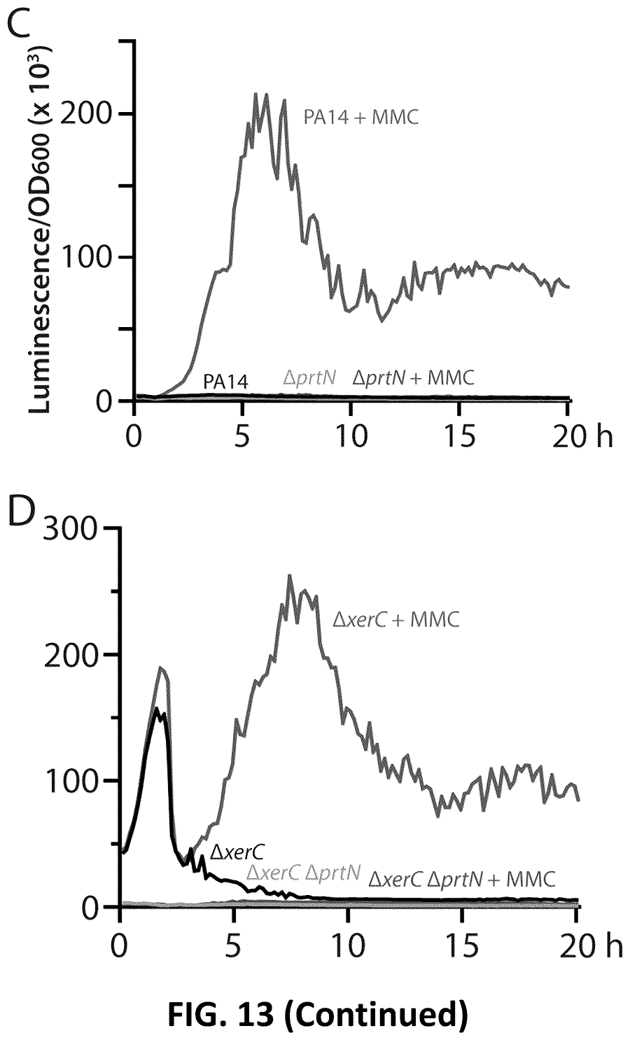

Example 1 demonstrated that the elevated basal expression of pyocins in ΔxerC strains required the pyocin expression activator PrtN, demonstrating that both the canonical (RecA-dependent) and non-canonical (RecA-independent) pathways for pyocin expression share a requirement for PrtN to enact pyocin expression. Thus, it was queried whether PrtN was also required for the further stimulation of pyocin expression in cells undergoing DNA damage. When wild-type and ΔxerC cells deleted or not for prtN were challenged with 0.1 μg/ml MMC, it was seen that, as expected, deletion of prtN abolished the ability of MMC to stimulate pyocin expression in a wild-type background (FIG. 13, Panels A, C; note the different Y-axis scales for luminescence graphs). Furthermore, prtN deletion also fully abrogated pyocin expression in a ΔxerC background, even under MMC treatment (FIG. 13, Panels B, D). These results confirm the common requirement for PrtN for P. aeruginosa cells to activate pyocin expression, irrespective of which pathway is active.

### RecA-Independent Pyocin Stimulation is Heterogeneous at the Single-Cell Level

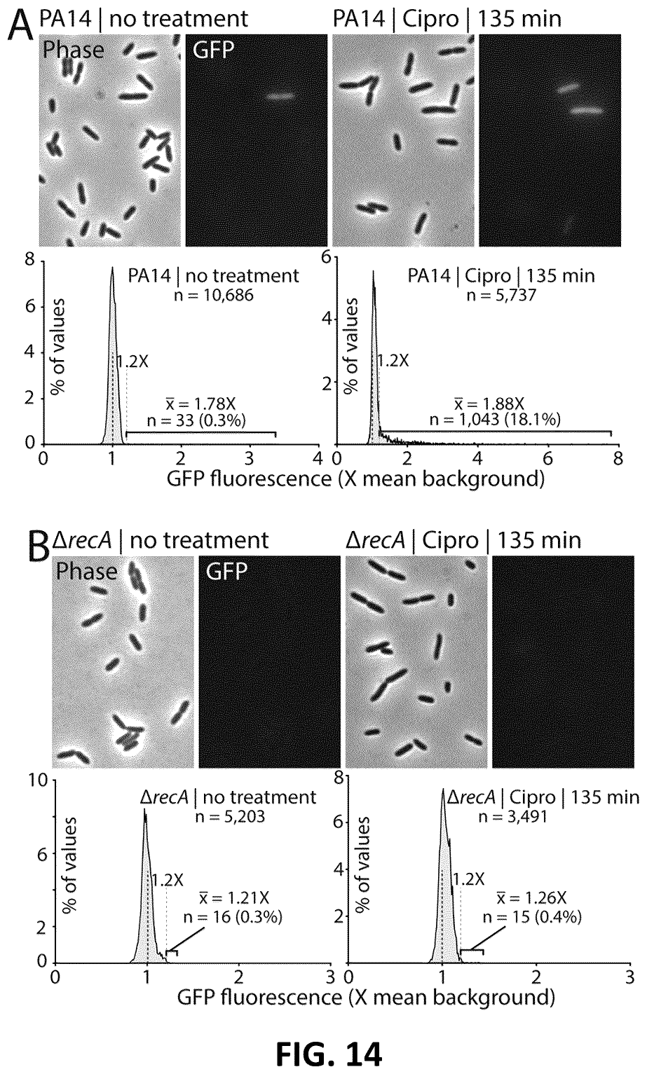

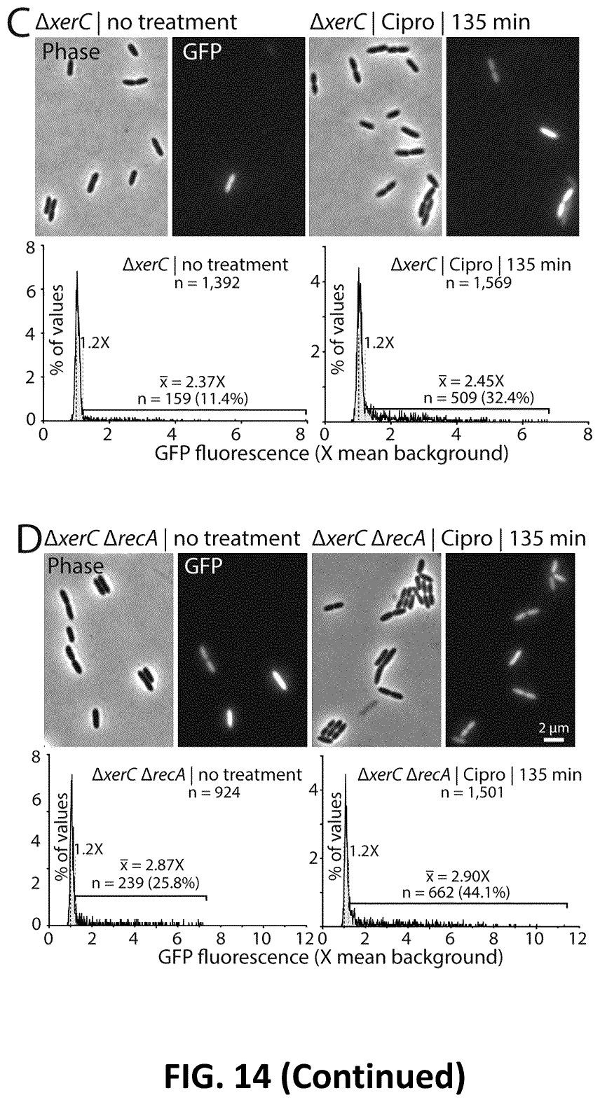

Pyocin expression, whether induced in wild-type cells via the canonical RecA-dependent pathway by ciprofloxacin or induced non-canonically in ΔxerC cells, shows strong heterogeneity at the single-cell level. Most cells showed undetectable pyocin expression (pyocin-OFF), whereas a subset of cells (pyocin-ON) displayed strong pyocin expression (visualized as a GFP transcriptional reporter driven by the PA14_07990 promoter at the beginning of the R/F pyocin gene cluster). It was further shown that pyocin-ON cells most often showed progressively increasing GFP fluorescence until cells explosively lysed due to the holin- and lysin-encoding genes in the R/F pyocin cluster. In the present Example, bulk assays showed extremely strong levels of pyocin expression when ΔxerC cells were treated with MMC or ciprofloxacin (FIGS. 11, 12). Such an increase in overall expression might be due either to a general increase in gene expression across all cells, or an increase in the portion of pyocin-ON cells within a population of mainly pyocin-OFF cells. It was thus queried whether heterogeneity in pyocin expression was preserved in wild-type or ΔxerC cells deleted or not for recA and treated or not with 0.03% ciprofloxacin for 135 minutes. Notably, this treatment concentration and duration minimally impacted cell growth in the bulk assays (FIG. 11, Panels A-B). The control strains were concordant with previous results: untreated wild-type PA14 cells showed very few (0.3%) GFP-positive cells that were relatively dim, whereas 135-min treatment with 0.03 μg/ml ciprofloxacin substantially increased the proportion of GFP-positive cells to 18.1% (FIG. 14, Panel A). Notably, even under ciprofloxacin treatment, the great majority of cells showed no detectable GFP fluorescence. Consistent with the bulk assay data, a ΔrecA strain also showed very few pyocin-ON cells, irrespective of ciprofloxacin treatment (FIG. 14, Panel B). The few cells that exceeded the threshold for GFP positivity were only just above the threshold (FIG. 14, Panel B), consistent with an overall lack of pyocin expression and hence a strict dependence on RecA for pyocin expression in a xerC⁺ genetic background.

In both ΔxerC and ΔxerC ΔrecA strain backgrounds, untreated cells showed the expected strong heterogeneity and substantially increased numbers of pyocin-ON cells (FIG. 14, Panels C-D). Treatment of either strain with ciprofloxacin markedly increased the proportion of pyocin-ON cells to at least ⅓ of the total cells observed without substantially changing their average GFP brightness; both strains still exhibited strong heterogeneity, with most cells showing no detectable GFP fluorescence (FIG. 14, Panels C-D). For both treated and untreated cells, more fluorescent cells and slightly greater average fluorescence in the ΔxerC ΔrecA double mutant were observed (FIG. 14, Panels C-D), clearly indicating that loss of RecA does not impair pyocin expression in ΔxerC cells.

### Inactivation of XerC Recombinase Activity Induces Pyocin Expression Solely Via RecA-Dependent Mechanisms

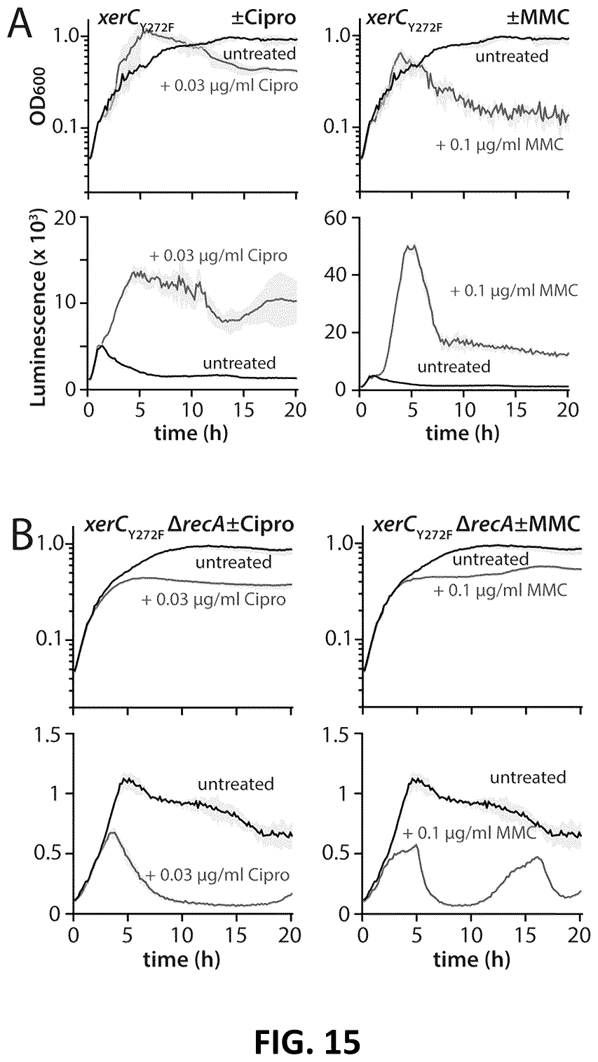

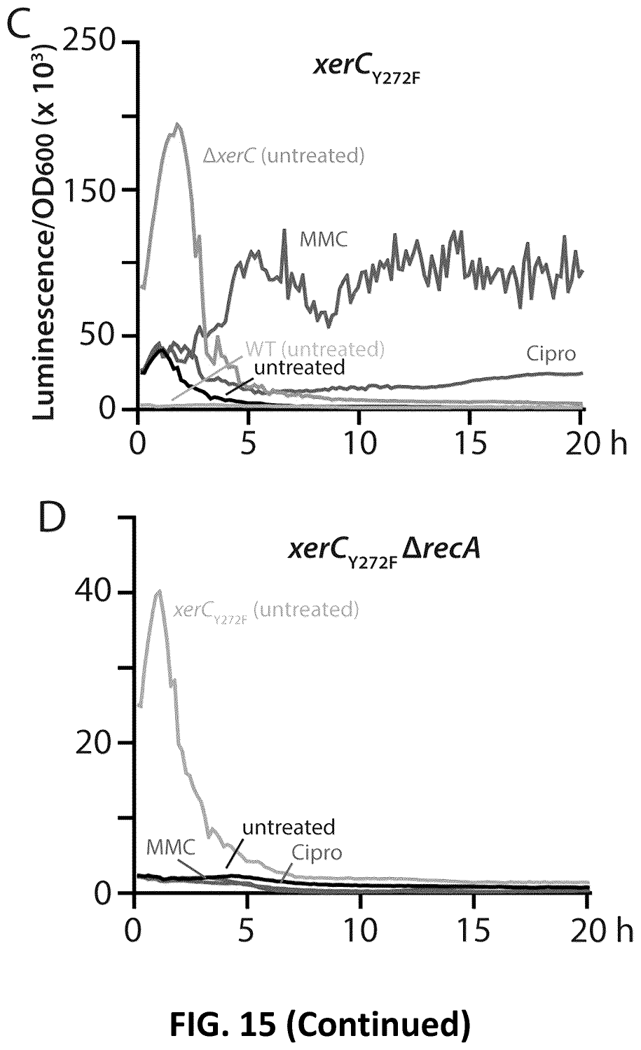

Because it was observed that both basal and DNA damage-induced pyocin expression in ΔxerC strains occurred independently of RecA, it was next asked whether the same were true of xerC_{Y272F} strains bearing only a recombinase-inactive version of XerC. Consistent with previous results, a xerC_{Y272F} strain showed an intermediate phenotype, with much greater pyocin expression than the wild type but roughly fivefold less than in a ΔxerC strain (FIG. 15, Panels A, C; note the different Y-axis scales for luminescence graphs). While pyocin expression in xerC_{Y272F} strains was stimulated by both ciprofloxacin and MMC, the degree of stimulation was modest, with only slightly more pyocin expression in ciprofloxacin-treated xerC_{Y272F} cultures than in ciprofloxacin-treated wild-type cultures (FIG. 15, Panel C, compare with FIG. 11, Panel C). Moreover, MMC treatment resulted in less expression in xerC_{Y272F} cultures than in the wild type (FIG. 15, Panel C, compare with FIG. 12, Panel C), a distinct departure from the dramatic increases in pyocin expression upon ciprofloxacin or MMC treatment of ΔxerC strains (FIG. 11, Panel D; FIG. 12, Panel D).

Then, the role of RecA in the elevated basal pyocin expression of xerC_{Y272F} strains and its further stimulation by ciprofloxacin or MMC were examined. Strikingly, deletion of recA not only abolished ciprofloxacin- and MMC-mediated stimulation of pyocin expression, it also fully abolished the elevated basal pyocin expression of the xerC_{Y272F} strain (FIG. 15, Panels B, D; note the different Y-axis scales from FIG. 15, Panel A for luminescence graphs), reducing it to levels indistinguishable from a ΔrecA strain (FIG. 11, Panel A). These data indicate that enzymatic inactivation of XerC recombinase activity provokes pyocin expression exclusively via the canonical RecA-dependent pathway. The more modest effect of ciprofloxacin and MMC on pyocin expression in a xerC_{Y272F} background is consistent with this interpretation, as the RecA-mediated DNA damage-response pathways typically stimulated by these agents would already be partially active. Furthermore, it was reproducibly observed that MMC- and especially ciprofloxacin-treated xerC_{Y272F} cultures initially outgrew both untreated control cultures (FIG. 15, Panel A, top row) and drug-treated wild-type cells (FIG. 11, Panel A; FIG. 12, Panel A). This phenomenon is in accord with “pre-activation” of RecA in xerC_{Y272F} cells providing a measure of protection against DNA damage-inducing drugs. Conversely, the greater OD of the xerC_{Y272F}ΔrecA culture than the xerC_{Y272F} parent under MMC treatment at later timepoints (compare FIG. 15, Panels A and B, top right panels) likely reflects reduced cell lysis because cells are no longer producing pyocins. Importantly, these results show that loss of XerC recombinase activity and absence of XerC induce pyocin expression via separate mechanisms. The RecA-independent pyocin stimulation in ΔxerC strains cannot be attributed to loss of XerC recombinase activity, thereby implying a second, RecA-independent function of XerC in the regulation of pyocin expression.

A Recombinase Inhibitor Peptide Inhibits Growth but does not Stimulate Pyocin Expression

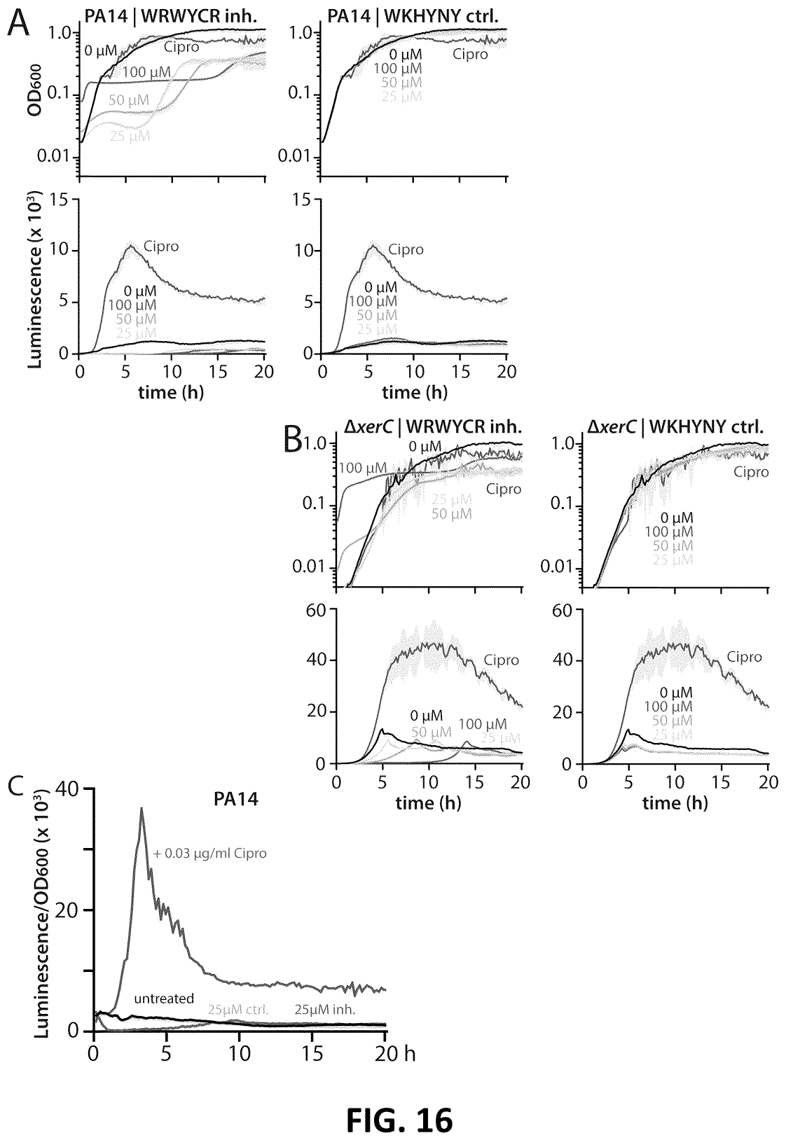

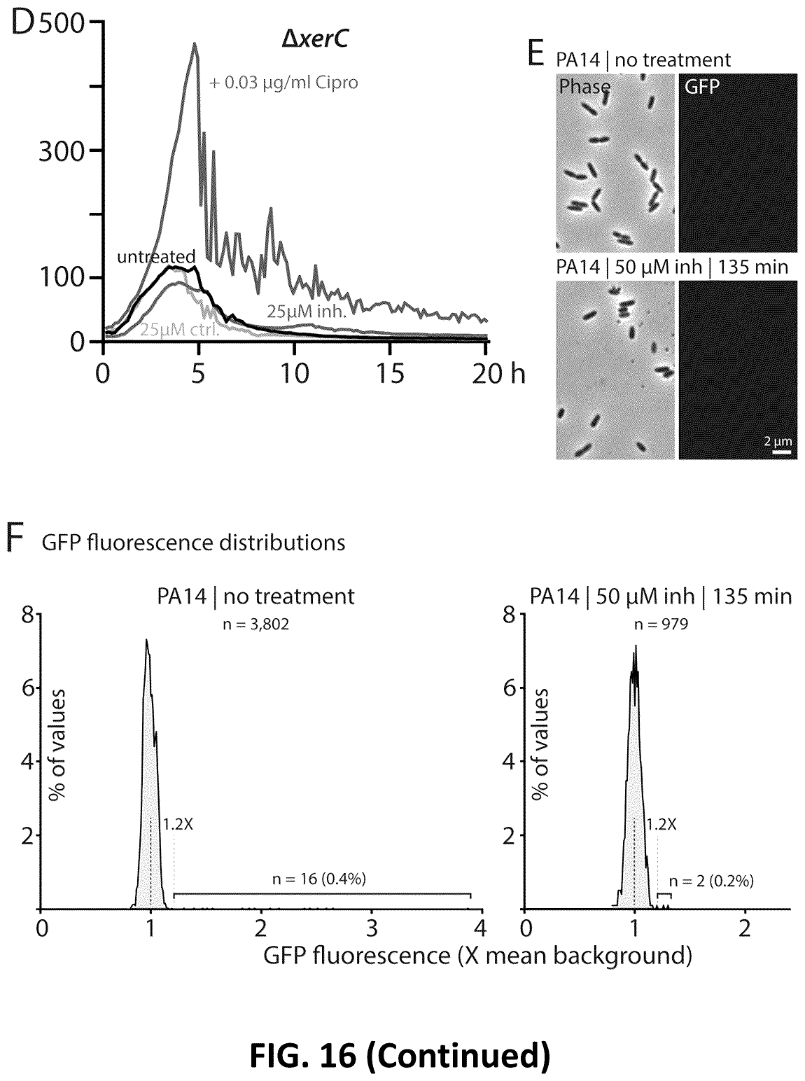

Given that both specific recombinase inhibition of XerC and full xerC deletion increase pyocin expression, albeit via different mechanisms, it was inquired whether a known tyrosine recombinase inhibitor could elicit pyocin expression. Drug treatment that inhibited XerC to stimulate pyocin expression would likely sensitize cells to fluoroquinolones like ciprofloxacin, imbuing recombinase inhibitors with potential therapeutic utility. Known inhibitors include hexapeptides that bind to Holliday junctions to achieve tyrosine recombinase inhibition; these inhibitors also inhibit growth of E. coli cells. However, these hexapeptide inhibitors have not been tested for their ability to stimulate pyocin production in P. aeruginosa. Wild-type cells were treated with the inhibitor WRWYCR (SEQ ID NO:3) at concentrations ranging from 25-100 μM, all of which markedly impaired P. aeruginosa growth (FIG. 16, Panel A). In contrast, the control hexapeptide WKHYNY (SEQ ID NO:4) showed no inhibition of growth at the same treatment concentrations (FIG. 16, Panel A). Neither the WRWYCR inhibitor nor the WKHYNY control elicited pyocin expression beyond the level of untreated cells (FIG. 16, Panels A, C), indicating that the inhibitor does not affect XerC in a way that provokes pyocin expression. Next, the same treatments were applied to ΔxerC cells, reasoning that this genetic background might sensitize cells to pharmacological inhibition of other recombinases. The growth of ΔxerC cells was inhibited by WRWYCR but not WKHYNY, with only minor differences from the wild type (FIG. 16, Panel B). Moreover, no differences in pyocin expression were observed under either inhibitor or control peptide treatment (FIG. 16, Panels B, D).

As a second test of the effect of inhibitor peptides, wild-type pyocin-GFP reporter cells treated with 50 μM of the WRWYCR inhibitor, a concentration that substantially inhibited growth, were also examined (FIG. 16, Panel A). The peptide-treated cells appeared morphologically similar to untreated cells, although a qualitative decrease in the number of dividing cells was observed (FIG. 16, Panel E), consistent with the growth inhibition measured in bulk (FIG. 16, Panel A). In accord with the bulk measurements, peptide treatment did not elicit GFP fluorescence (FIG. 16, Panel F), further supporting the conclusion that the peptide recombinase inhibitor WRWYCR does not stimulate pyocin production in P. aeruginosa.

### Subinhibitory Concentrations of Peptide Inhibitors do not Stimulate Pyocin Expression

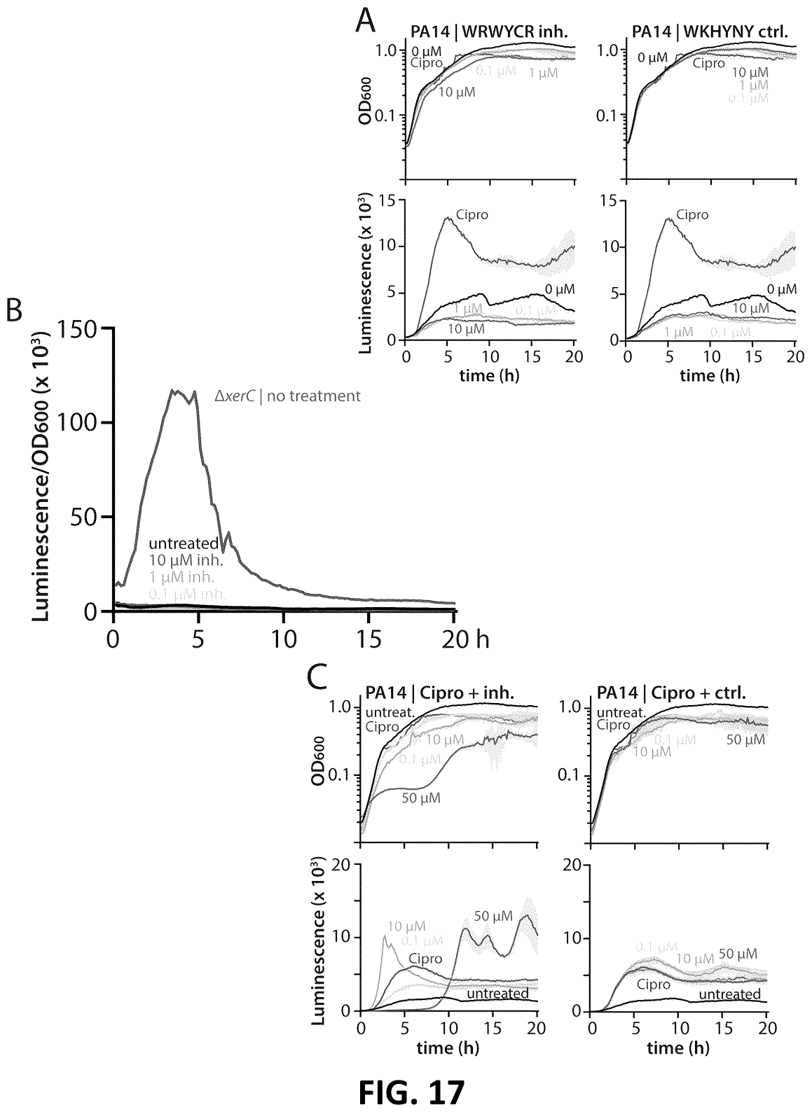

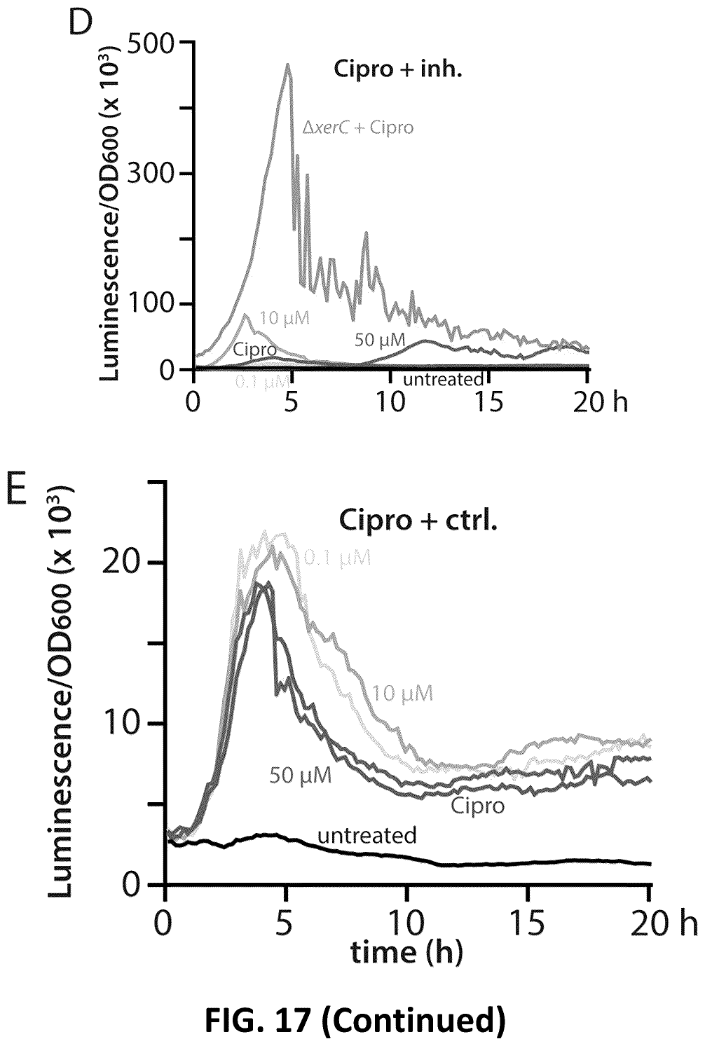

It was clear from the data that growth-inhibitory concentrations of the tyrosine recombinase inhibitor WRWYCR did not stimulate pyocin expression (FIG. 16). However, work with ciprofloxacin and MMC, which strongly elicit pyocin expression even at concentrations that do not fully inhibit cell growth, prompted examination of the effects of lower concentrations of hexapeptides that have only minor effects on growth. In these experiments, wild-type cells were treated with 0.1-10 μM of WRWYCR or the WKHYNY control peptide. Treatment with 10 μM inhibitor affected culture growth to a similar degree as 0.03 μg/ml ciprofloxacin, whereas lower concentrations of inhibitor had correspondingly smaller effects, as did the control peptide (FIG. 17, Panel A). Treatment with low concentrations of WRWYCR resulted in no increase in pyocin expression over untreated cells (FIG. 17, Panels A-B).

### Co-Administration of Peptide Inhibitors to Ciprofloxacin-Treated Cells Modestly Impacts Pyocin Expression

As a final test of the impact of recombinase inhibitor peptides on pyocin production by P. aeruginosa, different concentrations of inhibitor or control peptides were applied in combination with 0.03 μg/ml ciprofloxacin. Because pyocin expression in ΔxerC cells is sensitive to ciprofloxacin, it was reasoned that treatment with a fluoroquinolone antibiotic might sensitize cells to even mild recombinase inhibition, resulting in measurable changes to pyocin expression. As expected, treatment with the control peptide over a range of 0.1-50 μM impacted neither culture growth nor pyocin expression (FIG. 17, Panels C, E). However, co-treatment with the WRWYCR inhibitor at 10 or 50 μM, but not at 0.1 μM, yielded modest changes to the magnitude and timing of pyocin expression. A 10 μM concentration of inhibitor induced an earlier and higher peak of pyocin expression relative to ciprofloxacin alone (FIG. 17, Panels C-D) or inhibitor alone (FIG. 17, Panels A-B). Co-treatment with 50 μM inhibitor induced a later peak of pyocin expression, in accord with initial inhibition of growth (FIG. 17, Panel C). As with 10 μM inhibitor co-treatment, the peak of pyocin expression was greater than for either treatment alone (FIG. 17, Panels C-D, compare to FIG. 16, Panels A-B). These data indicate that recombinase inhibition by hexapeptides like WRWYCR can modestly impact pyocin expression in cells with an already-active SOS response. However, the slightly greater peak pyocin expression induced by addition of peptide inhibitor to ciprofloxacin remained several-fold lower than the peak observed for ciprofloxacin treatment of ΔxerC cells (FIG. 17, Panel D). Collectively, these results imply that peptide recombinase inhibitors do not substantially inhibit XerC in a manner leading to pyocin expression.

### Discussion

Four principal findings can be derived from this Example. First, the absence of XerC results not only in RecA-independent elevation of basal pyocin expression but also permits substantial additional stimulation of pyocin expression by ciprofloxacin or MMC (FIGS. 11-12). This additional stimulation is likewise RecA-independent despite being provoked by agents that cause DNA damage. Nonetheless, under all tested conditions, pyocin expression requires PrtN (FIG. 13). Second, heterogeneity in pyocin expression across individual cells is preserved even under very strong bulk expression (FIG. 14), consistent with a robust system to prevent widespread cell lysis. Third, specific inhibition of XerC recombinase activity induces pyocin expression only via the canonical RecA-dependent pathway (FIG. 15), implying that XerC has a second function in pyocin regulation that is separate from its recombinase activity. Finally, a previously characterized bacterial tyrosine recombinase inhibitor does not activate pyocin expression at either inhibitory or sub-inhibitory concentrations (FIGS. 16-17).

Our finding that deletion of xerC raises basal pyocin expression levels that can be further stimulated by ciprofloxacin or MMC helps to explain the previously observed hypersensitivity of ΔxerC cells to ciprofloxacin. It also raises additional questions with respect to the nature of the RecA-independent pathway for pyocin expression. RecA-independent induction of typically RecA-dependent pathways is not entirely without precedent. Expression of certain capsular polysaccharide synthesis regulators can induce RecA-independent lambda prophage induction in E. coli, and Mycobacteria have a well-studied RecA-independent DNA damage response that is regulated by proteasome accessory factors. Irrespective of the pathway, PrtN appears to be strictly required for pyocin expression (FIG. 13), implying that prtN expression can occur even without activated RecA-mediated cleavage of PrtR. Identifying the factors required for RecA-independent pyocin induction is an important future goal.

Our microscopic analysis showed that even under DNA damage-inducing conditions producing the strongest pyocin response, pyocin gene expression remained highly heterogeneous, with fewer than half of cells showing detectable expression (FIG. 14). Clearly, when inducing a gene cluster that encodes a lethal holin and lysin, a heterogeneous response can be advantageous, as non-expressing cells are protected from lysis. It will be interesting to uncover the basis for the strong heterogeneity of pyocin production, which remains unclear.

It was initially surprising to find that the modest elevation of pyocin expression in strains bearing the recombinase-inactive XerC_{Y272F} variant was mediated by RecA (FIG. 15). However, distinct mechanisms are concordant with the stronger pyocin expression seen in ΔxerC strains relative to xerC_{Y272F} strains. These results also imply that the presence of recombinase-dead XerC in cells provokes RecA activation in a way that the complete absence of XerC does not. Moreover, the absence of RecA-independent pyocin expression in xerC_{Y272F} cells indicates that XerC has a second function in pyocin regulation that is not affected by its enzymatic inactivation. One intriguing possibility warranting further investigation is that XerC also acts as a transcriptional regulator at sites other than the dif sites at which it cooperates with XerD to achieve recombination.

Because deletion of xerC leads to increased pyocin production and sensitizes cells to ciprofloxacin, a member of the clinically important fluoroquinolone class of antibiotics, it was considered important to test whether drug treatment could achieve a similar effect. To the inventors' knowledge, the only known inhibitors of bacterial recombinases are hexapeptides, which have primarily been characterized in E. coli. These inhibitors, the best known of which is WRWYCR (or wrwycr, constructed from D-amino acids), trap Holliday junctions (including intermediates in XerCD-mediated chromosome dimer resolution), can prevent prophage excision, and thereby inhibit bacterial growth. It was confirmed that WRWYCR, but not a previously described control hexapeptide, WKHYNY, effectively inhibited growth of the P. aeruginosa strains (FIG. 16). Neither the experiments disclosed herein nor previous work rules out the possibility that the control WKHYNY peptide is simply not taken up by bacterial cells. Nonetheless, given that genetic inactivation of XerC (XerC_{Y272F}) induced RecA-dependent pyocin expression (FIG. 15) and that DNA damage stemming from wrwycr-mediated inhibition induced the SOS response in E. coli, it was expected that general recombinase inhibition might induce pyocins. However, no induction was observed under growth-inhibitory levels of peptide treatment (FIG. 16). Because it was noticed that relatively low, sub-lethal concentrations of ciprofloxacin (0.03 μg/ml) resulted in more frequent pyocin-ON cells that the higher concentrations (1 μg/ml) used previously, lower concentrations of inhibitor peptide that had only weak effects on bacterial growth were also tested. Pyocin induction was never observed, confirming that recombinase inhibition via Holliday junction trapping does not, on its own, induce pyocin expression. Even in combination with ciprofloxacin treatment, inhibitor peptides had only a modest impact on pyocin expression and timing (FIG. 17).

Collectively, these results highlight the existence of an alternative, RecA-independent but DNA damage-inducible pathway for pyocin expression that was only observed in xerC-deleted strains. Further, these findings imply that P. aeruginosa is capable of sensing DNA damage even without RecA. As such, pharmaceutical inhibition of XerC may be achieved to activate the alternative pathway and sensitize P. aeruginosa to fluoroquinolone antibiotics.

### Materials and Methods

### Strains and Growth Conditions

Escherichia coli SM10 and Pseudomonas aeruginosa PA14 were grown in Luria-Bertani (LB) Lennox broth (10 g/L tryptone, 5 g/L yeast extract, 5 g/L NaCl) or on LB agar plates fortified with 1.5% Bacto agar at 37° C. When appropriate, 25 μg/ml irgasan (to specifically select for P. aeruginosa) plus 75 μg/ml tetracycline, 25 μg/ml irgasan plus 75 μg/ml gentamicin, 25 μg/ml tetracycline, or 20 μg/ml gentamicin was added to liquid or solid media. P. aeruginosa was also selected over E. coli for some strains by growth on VBMM containing citrate as the sole carbon source. The strains used in this Example are listed in Tables 1-3. Markerless deletions were generated using the pEXG2 vector with counterselection on no-salt LB plates containing 15% sucrose and were screened by colony PCR for the presence of deletions. Reporter strains were constructed by integration of the mini-CTX-1-gfp vector at the neutral chromosomal attB locus. Modes of strain and plasmid construction are given in the Supplemental Materials. Strains deleted for recA were additionally phenotypically screened for their inability to enact generalized recombination by failure to generate gentamycin-resistant EXG2 transconjugants.

### Growth Curve and Kinetic Luciferase Assays

Strains of interest were grown on LB plates overnight, and single colonies were inoculated into LB liquid broth with appropriate antibiotics and grown overnight with shaking at 37° C. Strains were then diluted 1,000-fold into fresh LB medium and grown to early exponential phase (2-4 hours). The cultures were then mixed in 1.5-ml microfuge tubes with stocks of ciprofloxacin, mitomycin C (both in sterile water), or hexapeptides (in DMSO) at >50× the final concentration and aliquoted (200 μl) into wells of a clear-bottomed, opaque white 96-well plate to generate technical replicates (3-4 per biological replicate). The plate was incubated in a BioTek Synergy H1 plate reader (BioTek, USA) at 37° C. for 20 h with double-orbital shaking. OD₆₀₀ and luminescence (gain=135, integration time, 1 sec) measurements were obtained every 10 minutes. At least 3 biological replicates were assayed for each combination of strain and condition. Results were analyzed in MS Excel and plotted using GraphPad Prism.

### Fluorescence Microscopy

Strains of interest were grown in 3 mL of LB liquid broth with appropriate antibiotics overnight. The cultures were then diluted 1,000-fold in fresh LB and grown to early exponential phase (3-4 hours). The cultures were split, and 0.03 μg/ml ciprofloxacin was added (or not) to cells and incubated for a further 135 min before imaging. Cells were immobilized by spotting 0.5 μL of the growing culture onto an LB-agarose pad and covering with cover glass. Imaging was immediately performed using a Nikon Eclipse Ti inverted fluorescence microscope with a Photometrics Prime 95B sCMOS digital camera, a Lumencor SOLA SE II 365 LED Light Engine, and an OKO temperature-controlled enclosure. Cell images were captured at 100× magnification in both phase and GFP channels. For quantification of GFP-positive cells, images were analyzed as in the prior work using the MicrobeJ plugin for ImageJ, segmenting on phase-contrast and taking the mean GFP values of the corresponding fluorescence images. Segmentation was performed with default values except that a minimum and maximum areas of 100 and 400 px were used, and circularity was delimited from 0-0.9. For options, “exclude on edges,” “shape descriptors,” “segmentation,” and “intensity” were selected. A threshold of 1.2 times the average background fluorescence was selected to denote GFP positivity, as 100% of PA14 cells without a GFP reporter fell below this threshold, which was approximately 5.5 standard deviations above the mean fluorescence of reporter-free cells. Analyses were conducted using MS Excel and plotted using GraphPad Prism.

### Example 3

Based on the data presented in Examples 1-2, a screening method was developed to identify putative anti-pseudomonal agents from a library or collection of small-molecule compounds. The library or collection of compounds are screened by culturing with a P. aeruginosa strain that has been genetically engineered to be recA deficient and to include a transcriptional reporter joined to a pyocin gene cluster promoter, and then identifying candidate agents when expression of the transcriptional reporter is increased compared to a negative control.

To screen for pyocin-stimulating small-molecule compounds, the US National Cancer Institute's Diversity Set VI is used (NCI, Bethesda, MD), representing approximately 1,500 structurally diverse compounds. The test strain is Pseudomonas aeruginosa PA14 ΔrecA attB::CTX-1-P₀₇₉₉₀-lux. This strain harbors a luminescent reporter for the PA14_07990 gene, which is a member of the pyocin gene cluster and hence reports on pyocin expression. This reporter has previously been used extensively by the Cabeen lab to detect pyocin expression in different strains, and it is a reliable proxy for pyocin production. The test strain is also deleted for the recA gene, which is required for canonical, DNA damage-induced pyocin production. As such, the test strain is not expected to respond to DNA damaging agents but will only respond to drugs that can induce RecA-independent pyocin production. Such an effect phenocopies xerC deletion, which induces RecA-independent pyocin production.

The Diversity Set VI is added at different final concentrations (1 mM, 100 μM, 10 μM) to early exponential-phase cultures (200 μl volume) of the test strain in a 96-well clear-bottomed white polystyrene plate. A ΔxerC ΔrecA reporter strain acts as a positive control, whereas an untreated culture of the test strain serves as a negative control. Each 96-well plate is incubated with shaking in a Bio-Tek Synergy H1 plate reader, and the OD₆₀₀ and luminescence is measured every 10 minutes to detect pyocin expression. Candidate agents present in the Diversity Set VI that stimulate pyocin expression over the negative control are identified as putative anti-pseudomonal agents and subjected to further screening (such as, but not limited to, screens to determine if the putative agents inhibit XerC and/or XerD expression and/or activity).

Certain drugs that induce RecA-independent pyocin expression may target XerC or XerD; however, this screen will uncover any target whose inhibition induces RecA-independent pyocin expression.

### Example 4

The screen described in Example 3 is repeated with various other libraries, including (but not limited to) one or more of the following: Pharmakon collections such as (but not limited to) the Pharmakon-1760 collection (MicroSource Discovery Systems, Inc., New Milford, CT); ChemBridge libraries (ChemBridge Corp, San Diego, CA); various published libraries available from the Derek Tan lab at Memorial Sloan Kettering Cancer Center (New York, NY); and the like.

As stated above in Example 3, the screens of Example 4 uncover putative anti-pseudomonal agents that stimulate pyocin expression, and these putative agents may be subjected to further screening (such as, but not limited to, screens to determine if the putative agents inhibit XerC and/or XerD expression and/or activity).

Certain drugs that induce RecA-independent pyocin expression may target XerC or XerD; however, this screen will uncover any target whose inhibition induces RecA-independent pyocin expression.

### Example 5

Pseudomonas aeruginosa is a versatile bacterial species that is an opportunistic human pathogen. It competes with other P. aeruginosa strains by producing extracellular killing complexes such as pyocins, which are generally produced during DNA damage, including damage induced by fluoroquinolone antibiotics, DNA-damaging agents, or other SOS-inducing conditions. P. aeruginosa strains lacking the recombinase enzyme XerC strongly produce pyocins independent of SOS activation and are also hypersensitive to fluoroquinolone antibiotics. Moreover, treatment of xerC deletion strains with fluoroquinolones results in hyperproduction and release of pyocins from the bacterial cell.

The compositions, kits, and methods of certain non-limiting embodiments of the present disclosure exploit the characteristics of a xerC deletion mutant strain of P. aeruginosa, which exhibits enhanced production of pyocin (P. aeruginosa-killing protein complexes) and increased sensitivity to fluoroquinolone antibiotics. Compositions comprising chemical inhibitors of XerC (such as, but not limited to, those identified by the screening methods of Examples 3-4) are produced and utilized alone or in a combinatorial therapy with fluoroquinolone antibiotics as a therapy against P. aeruginosa infection. The chemical inhibitors that inhibit XerC mimic P. aeruginosa xerC deletion mutants and thus can be utilized alone or in combination with fluoroquinolone antibiotics to stimulate the production of pyocin and increase bacterial sensitivity to fluoroquinolone antibiotics.

Therefore, combination treatment with a XerC inhibitor and a fluoroquinolone antibiotic induces a primary and a secondary effect. The primary effect, which would always occur, would be to enhance antibiotic-mediated cell killing and pyocin production and release. The secondary effect, which would occur if pyocin-sensitive cells were in the vicinity, would be that the released pyocins would kill nearby pyocin-sensitive P. aeruginosa cells. Thus, combination treatment with at least one XerC inhibitor and at least one fluoroquinolone may provide an effective therapy against P. aeruginosa infections.

The treatment method involves exploiting the enhanced production of pyocins (cell killing proteins) and the increased sensitivity to fluoroquinolone antibiotics of xerC deletion mutant strains of P. aeruginosa. The combinatorial use of XerC inhibitors and fluoroquinolone antibiotics represents a potential new therapy against P. aeruginosa infection.

The method involves producing mutant strains of P. aeruginosa with xerC deletion and capable of producing pyocins (as described in Examples 1-2). Production of pyocin proteins in the absence of xerC is independent of RecA and the SOS response. The method also includes inducing pyocin production by administering at least one fluoroquinolone antibiotic to P. aeruginosa stains with or without the xerC deletion.

The method involves producing a P. aeruginosa ΔrecA strain with pyocin-mediated lysis blocked (deletions of PA14_07990 and PA14_08160) and a chromosomal luciferase reporter for pyocin expression to screen for XerC inhibitors (as described in Examples 1-2). This strain emits light if pyocin expression is active.

The method involves producing a P. aeruginosa strain with such deletions as are necessary (at least ΔrecA Δpyocins ΔalpBCDE, and as described in Examples 1-2) to prevent pyocin-mediated cell death and maximize the sensitivity of the inhibitor screen.

The P. aeruginosa ΔrecA Δpyocins ΔalpBCDE strain as well as other deletion strains with a chromosomal luciferase reporter are screened for pyocin expression; these strains emit light if pyocin expression is active, even if no pyocins are produced. The goal is to make a strain that strongly induces pyocin expression but doesn't die as a result of that expression.

A library or collection of candidate agents is then screened for potential XerC inhibitors that interact with XerC in a way that results in fluoroquinolone sensitivity and pyocin production (light emission in the reporter strain; see Examples 3-4 for similar screening assay that can be used with the strains described above).

The subsequently identified XerC inhibitors can be used alone or in combination with fluoroquinolone in a combinatorial therapy that provides enhancement of fluoroquinolone sensitivity as well as increased production of pyocins in the targeted cells.

Thus, in accordance with the present disclosure, there have been provided compounds, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

1. A method of screening for an anti-pseudomonal composition, the method comprising the steps of:
contacting at least one candidate agent with a P. aeruginosa strain to form a mixture, wherein the P. aeruginosa strain has been genetically engineered to be recA deficient and to comprise a transcriptional reporter joined to at least a portion of a promoter for at least one gene of a pyocin gene cluster;
culturing the mixture under conditions that allow for expression of the transcriptional reporter; and
determining that the at least one candidate agent is a putative anti-pseudomonal agent when the expression of the transcriptional reporter is increased when compared to expression thereof in the absence of the at least one candidate agent.

2. The method of claim 1, wherein the pyocin gene cluster is a R/F pyocin gene cluster.

3. The method of claim 1, wherein the P. aeruginosa strain has been genetically engineered to inhibit pyocin production.

4. The method of claim 3, wherein the genetically engineered P. aeruginosa strain comprises deletion of at least one of PA14_07990 and PA14_08160.

5. The method of claim 1, wherein the genetically engineered P. aeruginosa strain is alpBCDE deficient.

6. The method of claim 1, wherein the genetically engineered P. aeruginosa strain is P. aeruginosa PA14 ΔrecA attB::CTX-1-P₀₇₉₉₀-lux.

7. The method of claim 1, wherein the transcriptional reporter is at least one of a luminescent compound and a fluorescent compound.

8. The method of claim 7, wherein the transcriptional reporter is selected from the group consisting of Green Fluorescent Protein (GFP), Yellow Fluorescent Protein (YFP), Red Fluorescent Protein (RFP), Cyan Fluorescent Protein (CFP), mCherry, mKate2, mPlum, a luciferase, and combinations thereof.

9. The method of claim 1, wherein the genetically engineered P. aeruginosa strain is xerC deficient, or wherein XerC is inactivated.

10. The method of claim 9, wherein the mixture is cultured in the presence of at least one DNA-damaging agent.

11. The method of claim 10, wherein the at least one DNA-damaging agent is mitomycin C.

12. The method of claim 1, wherein the method is a high throughput screening (HTS) method.

13. The method of claim 1, further comprising the step of assaying the mixture to determine if at least one of tyrosine recombinase XerC or XerD expression or activity is inhibited when compared to expression or activity thereof in the absence of the at least one candidate agent.

14. A method of screening for an anti-pseudomonal composition, the method comprising the steps of:
contacting at least one candidate agent with a P. aeruginosa strain to form a mixture, wherein the P. aeruginosa strain has been genetically engineered to be recA and pyocin deficient and to comprise a transcriptional reporter joined to at least a portion of a promoter for at least one gene of a R/F pyocin gene cluster;
culturing the mixture under conditions that allow for expression of the transcriptional reporter; and
determining that the at least one candidate agent is a putative anti-pseudomonal agent when the expression of the transcriptional reporter is increased when compared to expression thereof in the absence of the at least one candidate agent.

15. The method of claim 14, wherein at least one of:
the genetically engineered P. aeruginosa strain comprises deletion of at least one of PA14_07990 and PA14_08160;
the genetically engineered P. aeruginosa strain is alpBCDE deficient; and
the genetically engineered P. aeruginosa strain is P. aeruginosa PA14 ΔrecA attB::CTX-1-P₀₇₉₉₀-lux.

16. The method of claim 14, wherein the transcriptional reporter is at least one of a luminescent compound and a fluorescent compound.

17. The method of claim 16, wherein the transcriptional reporter is selected from the group consisting of Green Fluorescent Protein (GFP), Yellow Fluorescent Protein (YFP), Red Fluorescent Protein (RFP), Cyan Fluorescent Protein (CFP), mCherry, mKate2, mPlum, a luciferase, and combinations thereof.

18. The method of claim 14, further comprising the step of assaying the mixture to determine if at least one of tyrosine recombinase XerC or XerD expression or activity is inhibited when compared to expression or activity thereof in the absence of the at least one candidate agent.

19. The method of claim 14, wherein the method is a high throughput screening (HTS) method.

20. The method of claim 14, wherein the genetically engineered P. aeruginosa strain is xerC deficient, or wherein XerC is inactivated, and wherein the mixture is cultured in the presence of at least one DNA-damaging agent.

21. A composition, comprising:
at least one anti-pseudomonal agent determined by the method of claim 1.

22. The composition of claim 21, further comprising at least one fluoroquinolone antibiotic.

23. The composition of claim 22, wherein the at least one fluoroquinolone antibiotic is selected from the group consisting of balofloxacin, besifloxacin, cinoxacin, ciprofloxacin, clinafloxacin, danofloxacin, delafloxacin, difloxacin, enoxacin, enrofloxacin, fleroxacin, flumequine, garenoxacin, gatifloxacin, gemifloxacin, garenoxacin, grepafloxacin, ibafloxacin, levofloxacin, lomefloxacin, marbofloxacin, moxifloxacin, nadifloxacin, naldixic acid, nemonoxacin, norfloxacin, ofloxacin, orbifloxacin, oxalinic acid, pazufloxacin, pefloxacin, piromidic acid, pipemidic acid, prulifloxacin, rosoxacin, rufloxacin, sarafloxacin, sitafloxacin, sparfloxacin, temafloxacin, tosufloxacin, trovafloxacin, DX-619; a pharmaceutically acceptable salt or stereoisomer of any of the above; or any combination thereof.

24-34. (canceled)


SEQUENCE LISTING (4 sequences)

SEQ ID NO: 1 - protein, 44 aa, Escherichia coli
GDLRGVQELLGHANLSTTQIYTHLDFQHLASVYDAAHPRAKRGK

SEQ ID NO: 2 - protein, 52 aa, Pseudomonas aeruginosa
GDLRAVQELLGHADIATTQIYTHLDFQHLASVYDRAHPRAKRKGNADGGNDP

SEQ ID NO: 3 - protein, 6 aa, synthetic construct
WRWYCR

SEQ ID NO: 4 - protein, 6 aa, synthetic construct
WKHYNY